(19) United States
(12) Patent Application Publication
(10) Pub. No.: US 2017/0173537 A1
(43) Pub. Date: Jun. 22, 2017

(54) APPARATUS AND METHODS FOR FRACTIONATION OF BIOLOGICAL PRODUCTS

(71) Applicant: AGENCY FOR SCIENCE, TECHNOLOGY AND RESEARCH, Singapore (SG)

(72) Inventor: Peter Stanley GAGNON, Singapore (SG)

(21) Appl. No.: 15/124,342

(22) PCT Filed: Mar. 9, 2015
(86) PCT No.: PCT/SG2015/050031

§ 371 Date: Sep. 7, 2016

Related U.S. Application Data
(60) Provisional application No. 61/949,944, filed on Mar. 7, 2014.

(51) Int. Cl. B01D 61/58; C07K 1/34; C07K 16/00; B01D 61/14

(52) U.S. Cl. CPC B01D 61/58; B01D 61/142; C07K 1/34; C07K 16/00; B01D 2325/42; B01D 2311/25; B01D 2311/2626; B01D 2311/08; B01D 2315/16; B01D 2315/10; B01D 2311/04

(57) ABSTRACT

Methods and apparatus for purification of biological molecules using combinations of tangential flow filtration subunits and adsorption sub-units in a single apparatus where the subunits may be operated independently or in series and where the apparatus and methods are suitable for use in a wide range of formats distinct from conditions required for chromatographic processes such as conducting process steps using adsorption subunits for purification of a biological molecule in a contaminated sample without prior buffer equilibration.

### CROSS-REFERENCE TO RELATED APPLICATIONS

This application claims the priority of U.S. provisional patent application No. 61/949,944 filed Mar. 7, 2014, the disclosure of which is incorporated herein by reference in its entirety.

Embodiments disclosed herein relate to apparatuses and processes suitable for purification of proteins, polynucleotides, and virus particles.

### BACKGROUND

Purification of proteins, for example IgG monoclonal antibodies, can be encumbered by the low efficiency of capturing the desired protein on a chromatography column. The slow flow rate and low capacity of porous particle chromatography media can inflate the column volume, which inflates the process buffer volumes, which inflates process time. Flow-through chromatography methods have been considered as an alternative, where the chromatography media binds only contaminants, however in such cases capacity is also limiting, even with porous particle media packed in columns, and such methods are nonetheless slow. Membrane and monolith-based chromatography can sometimes overcome the problem of slowness, but their capacity is many times less than columns packed with porous particle columns so that they have ultimately proven to be less efficient. Precipitation techniques and filtration techniques are known, but have also proven unable to provide a column alternative with equivalent fractionation performance but higher efficiency. Besides being individually inefficient, sequential multi-step purification processes suffer additional loss of efficiency from the discontinuity between process steps, which ultimately imposes the burdens of extra water consumption, greater material expense, and longer process time. The same limitations apply to purification of virus particles, and purification of DNA.

Certain apparatuses and processes involving combinations of adsorption chromatography and single-flow tangential flow filtration are known. Pall Life Sciences has published product literature linking single-pass tangential flow filtration chromatography with multiple columns packed with the same media for performing continuous chromatography, where the single-pass tangential flow assembly fulfills the function of concentrating the product, (M. Schofield et al, Pall Life Sciences, 2014, Application note USD 302, Productivity and economic advantages of coupling single-pass tangential flow filtration to multi-column chromatography for continuous processing. O. Shinkazh et al (U.S. Pat. Nos. 7,988,859, 7,947,175) have described apparatuses and processes involving fluidized adsorption chromatography particles pumped through tangential flow filtration systems, also for continuous processing.

Harvest clarification methods that particularly reduce chromatin content are known (H. T. Gan et al, J. Chromatography A 191 (2013) 33-40; P. Gagnon et al, J. Chromatogr. A 1340 (2014) 68-78). They improve the overall quality of purification and in some cases reduce the number of column chromatography steps to achieve the required purity, but productivity of the purification process remains impaired and multiple column chromatography steps are still required.

### SUMMARY

In certain aspects, the invention provides a process for purifying a biological product from a preparation including the steps of providing an apparatus which has (a) multiple purification subunits including a tangential flow filtration subunit equipped with at least one porous membrane having pores with a porosity sufficient to retain practically all of the biological product, and one or more adsorption subunits; (b) multiple conduits connecting the multiple purification units and associated pumps and valves, thereby allowing cycling of the biological product through the apparatus according to multiple alternate configurations including (i) a first configuration for continuous flow such that the retentate line output of the tangential flow filtration subunit may be collected as recyclate and returned to the input of the tangential flow filtration unit, and (ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and, optionally, (iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit; and (c) conduits for supply of the preparation to the apparatus, preferably to the input of the tangential flow filtration unit. In certain aspects, such processes additionally include performing Step A comprising operating the apparatus according to the first configuration such that the biological product may cycle through the tangential flow filtration subunit one or more times while increasing the concentration of the biological product and reducing the levels of contaminants associated with the biological product; performing Step B comprising operating the apparatus according to the second configuration such that the biological product may cycle through the tangential flow filtration subunit and the adsorption subunit selected from the one or more adsorption subunits one or more times while reducing the levels of contaminants associated with the biological product; and, optionally, performing Step C comprising operating the apparatus according to the third configuration such that the biological product may cycle through the tangential flow filtration subunit and the adsorption subunit different from the adsorption subunit selected in the second configuration one or more times while reducing the levels of contaminants associated with the biological product.

In certain aspects, the invention provides an apparatus for purifying a biological product from a preparation where the apparatus includes (a) multiple purification subunits comprising a first tangential flow filtration subunit equipped with at least one porous membrane having pores with a porosity sufficient to retain practically all of the biological product and one or more adsorption subunits; (b) multiple conduits connecting the multiple purification units; (c) valves that direct flow through the multiple conduits and permit isolation of one or more of the multiple purification units from each other; (d) pumps configured to induce flow and control differential pressure within one or more portions of the apparatus; and (e) conduits for supply of the preparation to the apparatus, preferably to the input of the tangential flow filtration unit; wherein the multiple conduits and associated pumps and valves allow cycling of the biological product through the apparatus according to multiple alternate configurations including (i) a first configuration for continuous flow such that the retentate line output of the tangential flow filtration subunit may be collected as recyclate and returned to the input of the tangential flow filtration unit, (ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and, (iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit.

### BRIEF DESCRIPTION OF THE DRAWINGS

The following Figures are provided to support clear explanation of the features of the disclosed apparatus and its applications. It is to be understood that these Figures are illustrative only and not limiting in any way to the specific configurations and applications of the apparatus as a whole.

FIG. 1 shows one configuration that embodies all certain functional features of an apparatus of the invention. The vessel indicated by the number 01 contains the product preparation to be purified. The vessel indicated by the number 02 contains a buffer that can be used to equilibrate the adsorption subunit (while only one buffer-containing vessel is shown, multiple buffers may be supplied as shown with respect to 02). 03 represents a recirculation vessel. 04 represents a filtrate (waste) vessel. 10 represents a balance so that the weight of the recirculation vessel can be known as an indicator of the contained volume of liquid at any point in time. 20 represents a valve for directing flow. 50, 51, 52, and 53 represent pumps. 30 represents a pressure sensor. 100 represents a tangential flow filtration subunit. 200 represents an adsorptive subunit. The adsorptive subunit in the figure represents a physical configuration that employs axial flow, such as would be the case with a column packed with chromatography particles, or a monolith, or a so-called membrane adsorber. It will be evident that a controller may be used to integrate pressure and other sensor signals to control differential pressures through various portions of the apparatus in order to support processes unique to such an apparatus. Numbered features described with respect to any of FIGS. 1-9 apply to features having the same numbers in all of FIGS. 1-9.

FIG. 2 highlights a flow path while the product preparation is being concentrated by the tangential flow filtration subunit. During concentration, small contaminants are eliminated though the pores in the tangential flow filtration module. The choice to concentrate is optional and the degree of concentration is optional, but the ability to concentrate the product is an aspect of the apparatus.

FIG. 3 shows a flow path while the buffer composition is being changed by diafiltration (buffer exchange) through the tangential flow filtration subunit. The choice to diafilter is optional, the extent of diafiltration is optional, and the chemical characteristics of the endpoint buffer are optional, but the ability to diafilter the preparation is an aspect of the apparatus.

FIG. 4 highlights a flow path with both the tangential flow filtration and adsorption subunits in line. This flow path can begin before, during, or after an optional buffer exchange. The ability of the tangential flow filtration module to retain the desired product permits the preparation to be cycled more than once through the adsorption subunit. This permits the adsorption subunit to be equilibrated to binding conditions coincident with buffer exchange. It also permits the preparation to be exposed initially to the adsorption subunit under conditions that prevent binding of contaminants to the adsorption subunit so that they can be removed through the pores in the tangential flow filtration module. This conserves the capacity of the adsorption subunit for binding contaminants when the buffer conditions later promote such binding. Small soluble contaminants continue to be eliminated through the pores in the tangential flow filtration membrane for the entire duration that the adsorption subunit is in line. Product concentration is maintained at the desired level throughout the process by controlling the balance of buffer input and fluid flux through the tangential flow filtration module.

FIG. 5 highlights a flow path while the adsorption subunit is being rinsed with clean buffer to recover the desired product. Product concentration is maintained at the desired level throughout the process by controlling the balance of input buffer and fluid flux through the tangential flow filtration module.

FIG. 6 shows a flow path with the adsorption subunit off-line, during rinsing of the tangential flow filtration module with clean buffer to recover the desired product. The buffer may optionally be exchanged at this step.

FIG. 7 shows a variation of FIG. 1 including an external adsorptive chromatography (201) so that the preparation may be further purified without requirement of performing a separate step on a separate chromatography system. The system further includes a collection vessel (5) for the purified product. The flow path may also be fitted with a sensor or sensor array on the outboard side of the adsorption subunit.

FIG. 8 shows a variation of FIG. 1 in which an additional tangential flow filtration module is included. Such a module provides the capability to use a first tangential flow filtration module to perform initial concentration of the preparation and to retain the product during the first adsorption step, then switch to the second tangential flow filtration module to avoid the potential for contaminants adsorbed to the first tangential flow module to leach into the preparation at later process stages. Membrane composition and porosity within the two tangential flow filtration modules might be the same, or different.

FIG. 9 shows a more complex apparatus based on FIG. 1 that differs by including three axial flow adsorption subunits 200, 201, 202. Other configurations could include 2, or 4, or more adsorption subunits, where the individual adsorption subunits could all employ an axial flow format, or all employ a tangential flow format, or a combination of formats. 05, 06, and 07 represent optional additional buffers or other desired materials.

FIG. 10 shows a partial flow path from an apparatus including a single tangential flow filtration subunit and a single adsorptive chromatography subunit, with the adsorptive chromatography subunit plumbed so that it can be placed in series with the tangential flow filtration subunit. 01 represents fluid inputs from buffers, sample and/or recyclate. 02 represents fluid output to recyclate. 03 represents permeate. 10 represents valves to control flow distribution. 100 represents a tangential flow filtration subunit. 200 represents an adsorption subunit. Numbered features described with respect to any of FIGS. 10-14 apply to features having the same numbers in all of FIGS. 10-14; reference numbers for features shown in FIGS. 10-14 are distinct from those of FIGS. 1-10 and 15. This configuration shown in FIG. 10 is an embodiment adequate to support applications such as those described in Examples 1, 2, 5, 8-13, and 15-22.

FIG. 11 shows a partial flow path from an apparatus including a single tangential flow filtration subunit and two adsorptive chromatography subunits, with the adsorptive chromatography units plumbed in parallel with respect to each other but so that either one can be in series with the tangential flow filtration subunit at any time, or both can be off-line with respect to the tangential flow filtration subunit. 01 represents fluid inputs from buffers, sample and/or recyclate. 02 represents fluid output to recyclate. 03 represents permeate. 10 represents valves to control flow distribution. 100 represents tangential flow filtration subunit. 200 represents an adsorption subunit. 201 represents a second adsorption subunit. This configuration permits the apparatus to mediate application of two distinct adsorptive methods to remove contaminants in situations where each adsorbent requires different operating conditions. This configuration shown in FIG. 11 is an embodiment adequate to support applications such as those described in Examples 3, 4, 6, 7, 24, and 25.

FIG. 12 shows a partial flow path from an apparatus including a single tangential flow filtration subunit and two adsorptive chromatography subunits, with the adsorptive chromatography units plumbed in series with respect to each other so that they can be placed together in series with the tangential flow filtration subunit. 01 represents fluid inputs from buffers, sample and/or recyclate. 02 represents fluid output to recyclate. 03 represents permeate. 10 represents valves to control flow distribution. 100 represents tangential flow filtration subunit. 200 represents an adsorption subunit. 201 represents a second adsorption subunit. This configuration permits the apparatus to mediate application of two distinct adsorptive methods to remove contaminants in situations where both adsorbents can be accommodated by the same operating conditions. This configuration shown in FIG. 12 is an embodiment adequate to support applications such as those described in Example 14.

FIG. 13 show a partial flow path from an apparatus including two tangential flow filtration subunits and a single adsorptive chromatography unit, where the two tangential flow filtration subunits are plumbed in parallel with respect to each other, but either can be run in series with the adsorptive chromatography subunit. 01 represents fluid inputs from buffers, sample and/or recyclate. 02 represents fluid output to recyclate. 03 represents permeate. 10 represents valves to control flow distribution. 100 represents a tangential flow filtration subunit. 101 represents a second tangential flow filtration subunit. 200 represents an adsorption subunit. This configuration permits the apparatus to concentrate and/or buffer exchange the preparation through one tangential flow filtration subunit in advance of processing the preparation through the adsorptive chromatography subunit, and then a different tangential flow filtration subunit after, or during and after processing the preparation through the adsorptive chromatography subunit. The benefit of this configuration is that the tangential flow filtration unit first in contact with the preparation may become fouled and then leach contaminants back into the preparation after the adsorption step. By having two tangential flow filtration subunits, a first such subunit that becomes fouled can be put off line to suspend the possibility of it leaching contaminants back into a highly purified preparation.

FIG. 14 shows a partial flow path from an apparatus including a single tangential flow filtration subunit, a single adsorptive chromatography unit, and a terminal non-recirculating adsorptive chromatography subunit on the permeate line from the tangential flow filtration subunit. 01 represents fluid inputs from buffers, sample and/or recyclate. 02 represents fluid output to recyclate. 03 represents permeate. 10 represents valves to control flow distribution. 100 represents a tangential flow filtration subunit. 200 represents an adsorption subunit. 300 represents a terminal non-circulating adsorption subunit. This configuration permits the apparatus to conduct a terminal contaminant adsorption step as the preparation exits the apparatus through the permeate line.

FIG. 15 shows another alternative configuration including integrated buffer formulation. The box labeled 01 indicates a water input. The boxes labeled 02, 03, and 04 represent inputs for buffers or buffer concentrates. The box labeled 05 indicates the input for the unpurified protein preparation. The black circle labeled 06 represents a switch valve that controls which input source is pulled into the system by the pump 07b. The amount of water admitted through pump 07a particular supports the ability to dilute buffer concentrates. The pump labeled 7c, in combination with pumps 071 and 07b, controls operating pressure between 07a/07b and 07c. The groupings of black triangles labeled 08 represent 3-way valves. The pair of black triangles labeled 09 represents a 2-postion valve that may be closed or open. The box labeled 10 represents a retentate-recirculation vessel. The box labeled 11 represents a waste tank. The box labeled 12 represents a tank into which the purified protein may be collected. The line labeled 13 represents a recirculation loop. The box labeled 100 represents a tangential flow filtration subunit. The box labeled 200 represents an adsorption subunit. The box enclosed by a dotted line and labeled 201 represents an optional additional adsorption subunit. The open circle labeled 1000 represents a mixer. The box labeled 1001 represents a pressure sensor. The box labeled 1002 represents a gas exchange device or bubble trap. Open triangles indicate direction of flow in the various flow paths of the apparatus. Reference numbers for features shown in FIG. 15 are distinct from those of FIGS. 1-14.

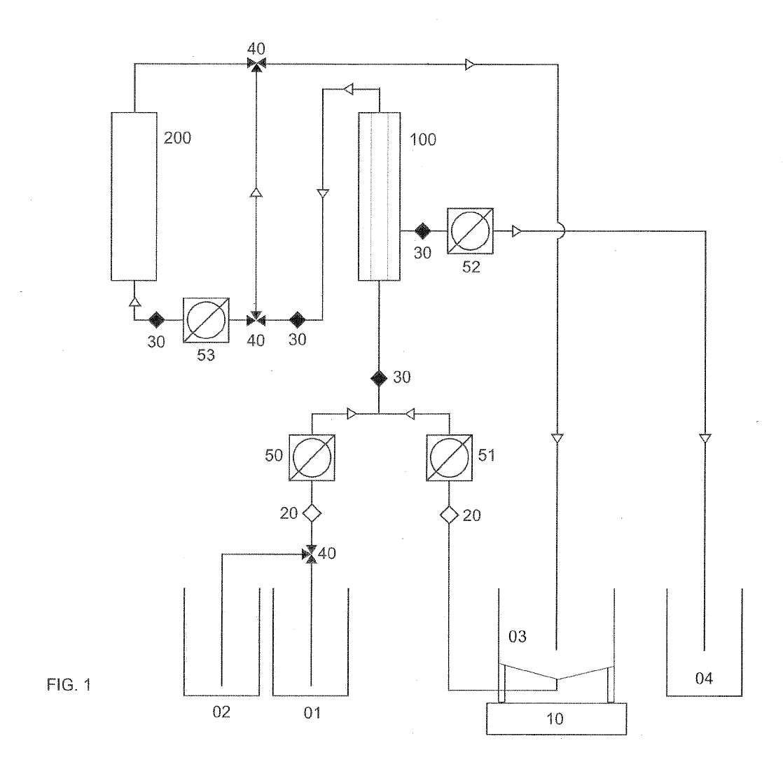

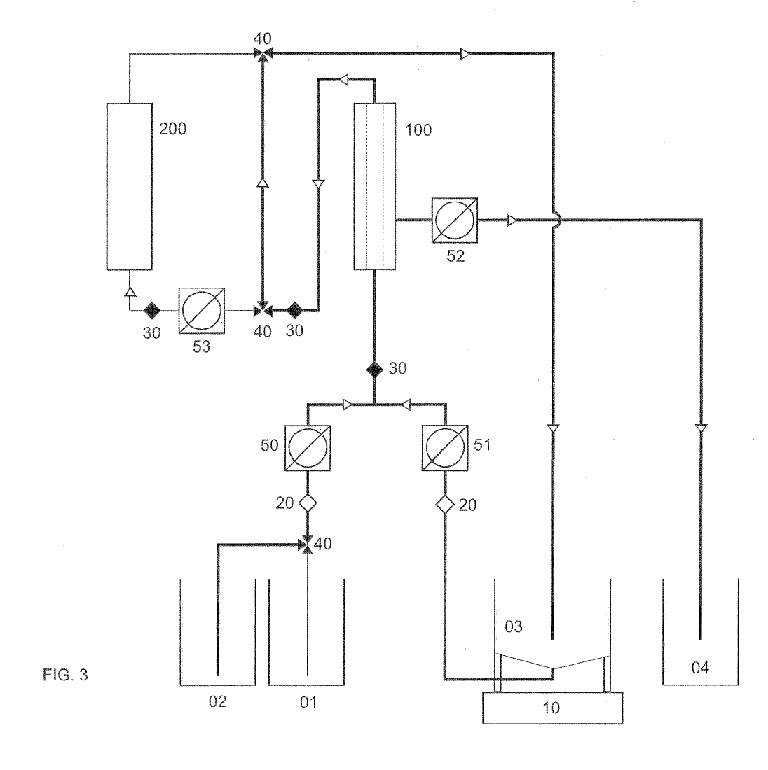

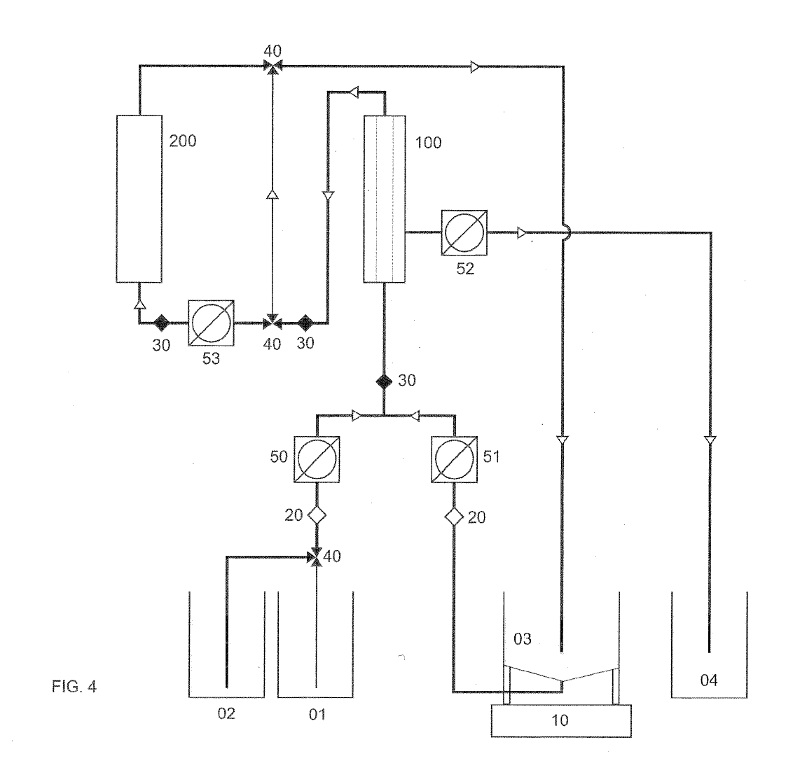

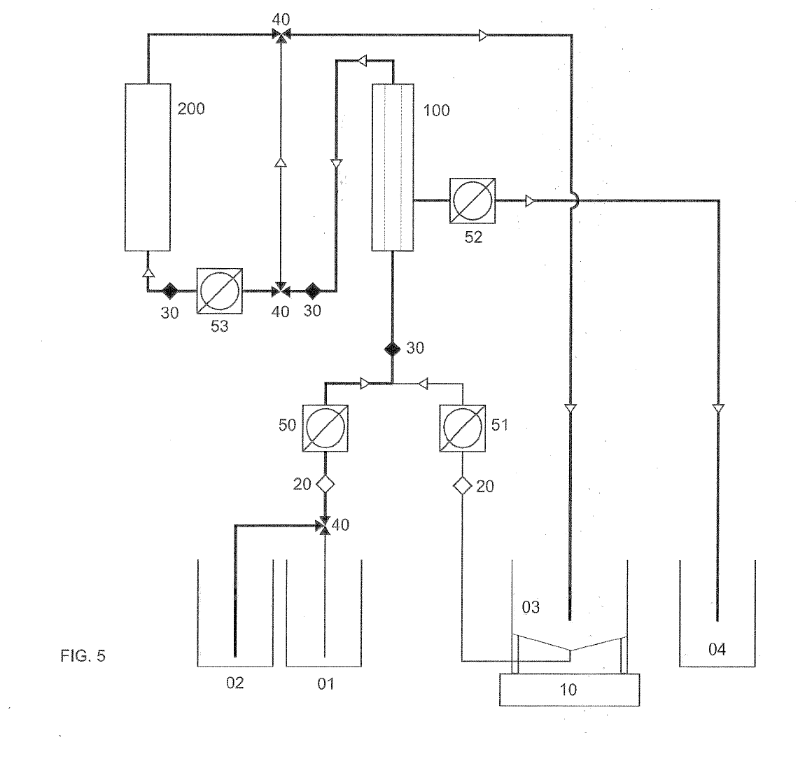

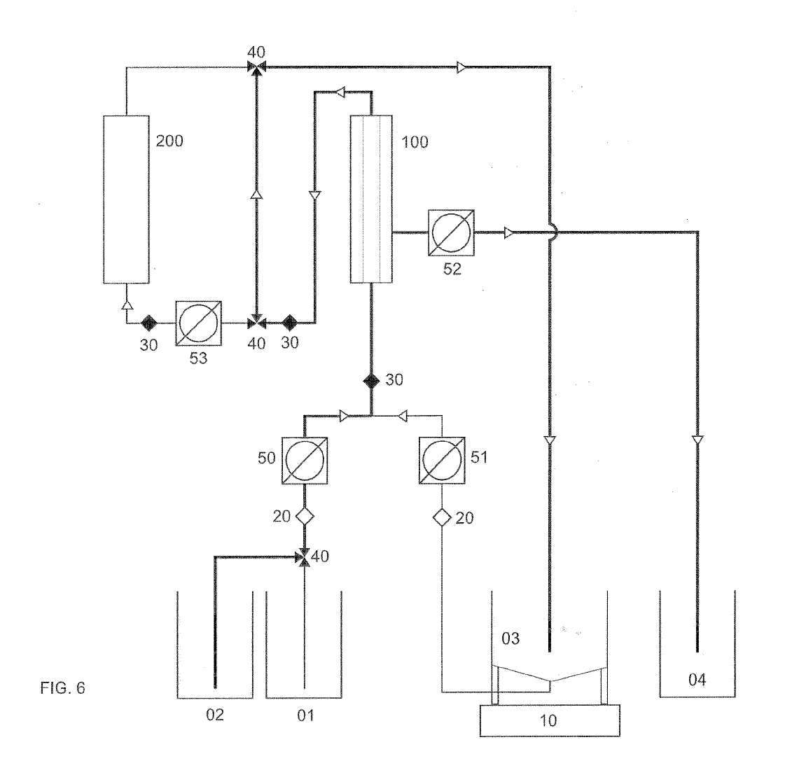

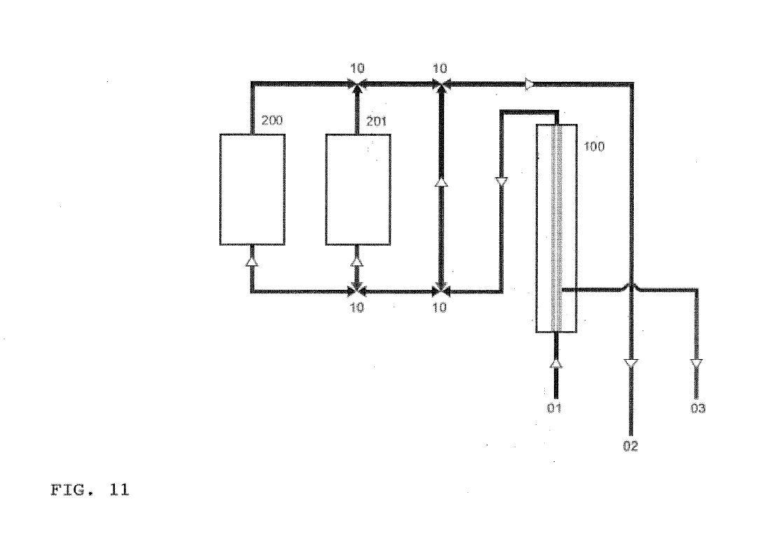

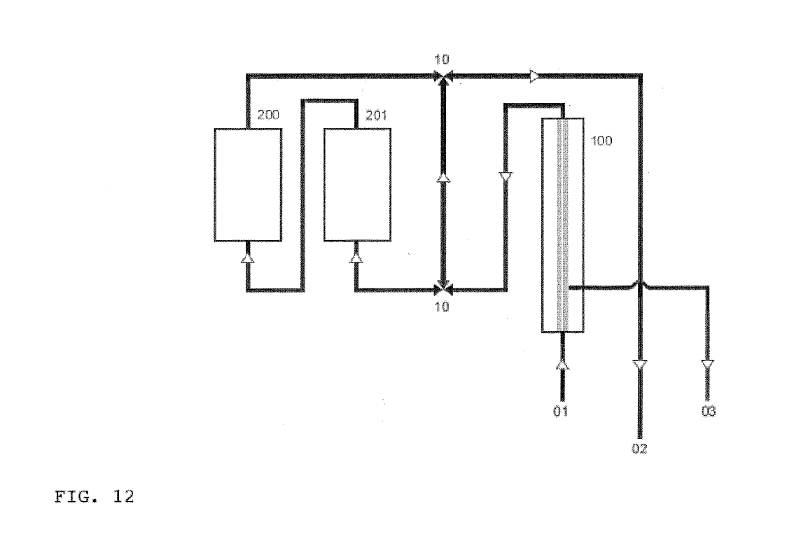

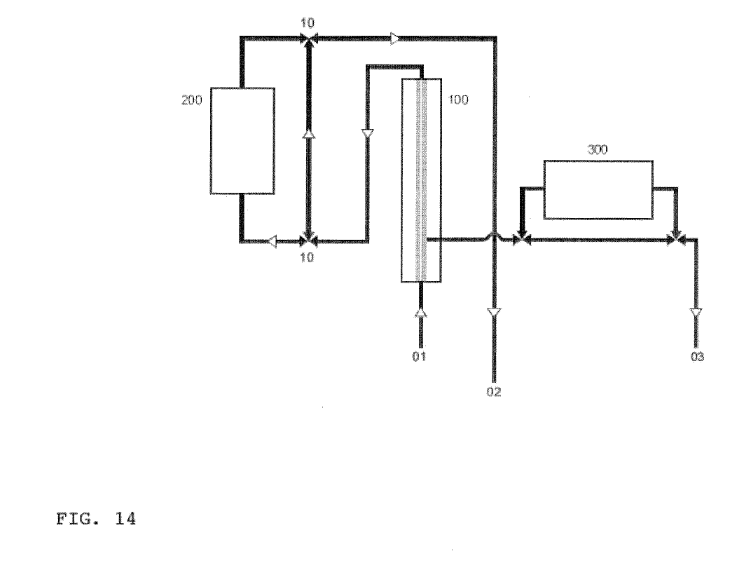

The embodiments depicted in and with reference to FIGS. 1-15 illustrates certain individual variations among apparatus and process configurations, but it will be apparent to the person of ordinary skill in the art, that various combinations, potentially including all of the individual features could be included in a single apparatus configuration.

### DETAILED DESCRIPTION

In certain aspects, the invention provides a process for purifying a biological product from a preparation including the steps of providing an apparatus which has (a) multiple purification subunits including a tangential flow filtration subunit equipped with at least one porous membrane having pores with a porosity sufficient to retain practically all of the biological product, and one or more adsorption subunits; (b) multiple conduits connecting the multiple purification units and associated pumps and valves, thereby allowing cycling of the biological product through the apparatus according to multiple alternate configurations including (i) a first configuration for continuous flow such that the retentate line output of the tangential flow filtration subunit may be collected as recyclate and returned to the input of the tangential flow filtration unit, and (ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and, optionally, (iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit; and (c) conduits for supply of the preparation to the apparatus, preferably to the input of the tangential flow filtration unit. In certain aspects, such processes additionally include performing Step A comprising operating the apparatus according to the first configuration such that the biological product may cycle through the tangential flow filtration subunit one or more times while increasing the concentration of the biological product and reducing the levels of contaminants associated with the biological product; performing Step B comprising operating the apparatus according to the second configuration such that the biological product may cycle through the tangential flow filtration subunit and the adsorption subunit selected from the one or more adsorption subunits one or more times while reducing the levels of contaminants associated with the biological product; and, optionally, performing Step C comprising operating the apparatus according to the third configuration such that the biological product may cycle through the tangential flow filtration subunit and the adsorption subunit different from the adsorption subunit selected in the second configuration one or more times while reducing the levels of contaminants associated with the biological product.

In certain aspects, the preceding embodiments provide a process wherein the apparatus provides for the first configuration, the second configuration and the third configuration and wherein each of Step A, Step B and Step C are performed.

In certain aspects, the preceding embodiments provide a process wherein the apparatus provides for the first configuration and the second configuration and further wherein Step A is performed followed by Step B and the purified biological product is obtained from the recyclate

In certain aspects, the preceding embodiments provide a process wherein the apparatus provides for the first configuration and the second configuration and further wherein Step A is performed followed by Step B, then Step A is performed again, and the purified biological product is obtained from the recyclate. In certain such embodiments, the recyclate containing the purified biological product is subjected to further purification by a chromatographic process. In other such embodiments, the recyclate containing the purified biological product is flowed to an additional adsorption unit and the biological product exiting such additional adsorption unit is not returned to the tangential flow unit before exiting the apparatus.

In certain aspects, the preceding embodiments provide a process wherein Step A is also performed between Step B and Step C. In certain such embodiments, the recyclate containing the purified biological product is subjected to further purification by a chromatographic process. In other such embodiments, the recyclate containing the purified biological product is flowed to an additional adsorption unit and the biological product exiting such additional adsorption unit is not returned to the tangential flow unit before exiting the apparatus.

In certain aspects, the preceding embodiments provide a process wherein the provided apparatus comprises a second tangential flow filtration purification unit equipped with at least one porous membrane having pores with a porosity sufficient to retain practically all of the biological product, and a second adsorption unit, the multiple conduits additionally allow cycling of the biological product through the apparatus according to multiple alternate configurations including the first configuration, the second configuration and (iv) a fourth configuration for continuous flow such that the retentate line output of the second tangential flow filtration subunit may be collected as recyclate and returned to the input of the second tangential flow filtration unit, and (v) a fifth configuration for continuous flow such that the retentate line output of the second tangential flow filtration connects to the input for the second adsorption subunit such that the output of such adsorption unit may be collected as recyclate and returned to the input of the second tangential flow filtration unit. In certain aspects, such processes additionally include performing Step D comprising operating the apparatus according to the fourth configuration such that the biological product may cycle through the second tangential flow filtration subunit one or more times while increasing the concentration of the biological product and reducing the levels of contaminants associated with the biological product; and performing Step E comprising operating the apparatus according to the fifth configuration such that the biological product may cycle through the second tangential flow filtration subunit and the second adsorption subunit one or more times while reducing the levels of contaminants associated with the biological product. In certain such embodiments, the apparatus provides for the first configuration, the second configuration, the fourth configuration and the fifth configuration and wherein each of Step A, Step B, Step D and Step E are performed.

In certain aspects, the preceding embodiments provide a process wherein chemical conditions during portions of Step B comprise one or more of the following: (i) conditions preventing adsorption of the majority of the components of the preparation, (ii) conditions preventing or suspending adsorption of the biological product, and (iii) conditions permitting adsorption of the biological product. In certain such embodiments, chemical conditions during portions of Step B comprise two or more of the following: (i) conditions preventing adsorption of the majority of the components of the preparation, (ii) conditions preventing or suspending adsorption of the biological product, and (iii) conditions permitting adsorption of the biological product.

In certain aspects, the preceding embodiments which include Step C provide a process wherein chemical conditions during portions of Step C comprise one or more of the following: (i) conditions preventing adsorption of the majority of the components of the preparation, (ii) conditions preventing or suspending adsorption of the biological product, and (iii) conditions permitting adsorption of the biological product. In certain such embodiments, chemical conditions during portions of Step E comprise two or more of the following: (i) conditions preventing adsorption of the majority of the components of the preparation, (ii) conditions preventing or suspending adsorption of the biological product, and (iii) conditions permitting adsorption of the biological product.

In certain aspects, the preceding embodiments which include Step E provide a process wherein chemical conditions during portions of Step E comprise one or more of the following: (i) conditions preventing adsorption of the majority of the components of the preparation, (ii) conditions preventing or suspending adsorption of the biological product, and (iii) conditions permitting adsorption of the biological product. In certain such embodiments, chemical conditions during portions of Step B comprise two or more of the following: (i) conditions preventing adsorption of the majority of the components of the preparation, (ii) conditions preventing or suspending adsorption of the biological product, and (iii) conditions permitting adsorption of the biological product.

In certain aspects, the preceding embodiments provide a process wherein during Step A chemical conditions are altered via diafiltration of the preparation through the tangential flow filtration subunit.

In certain aspects, the preceding embodiments provide a process wherein the porosity of the tangential flow subunits are substantially as large as possible while retaining substantially all of the biological product in the retentate during the process.

In certain aspects, the preceding embodiments having both a first and second tangential flow subunits provide a process wherein the membranes of the first and second tangential flow subunits are substantially the same. In other such embodiments, the membranes of the first and second tangential flow subunits have different chemical compositions and porosities that are substantially similar. In still other such embodiments, the membranes of the first and second tangential flow subunits have different chemical compositions and different porosities. In certain such embodiments, one membrane is a polyethersulfone membrane with 0.2 micron pores and the other membrane is a cellulose membrane with pore sizes corresponding with a globular protein with a mass of 30 kDa.

In certain aspects, the preceding embodiments provide a process wherein one or more membranes within one or more of the tangential flow filtration subunits has adsorptive surface characteristics such that it would be suitable for performing adsorption chromatography.

In certain aspects, the preceding embodiments provide a process wherein a physical format of the membrane within the tangential flow filtration subunit is selected from the group consisting of a sheet, a wound (rolled) sheet, a hollow fiber, and combinations thereof.

In certain aspects, the preceding embodiments provide a process wherein a physical format of the adsorptive subunits is selected from the group consisting of a column packed with adsorptive particles; a monolith, one or more adsorptive membranes, one or more sheets or one or more hollow fibers, and combinations thereof.

In certain aspects, the preceding embodiments provide a process wherein an adsorptive mechanism employed by the adsorptive subunits are independently selected from the group consisting of electrostatic interaction, hydrophobic interaction, pi-pi binding, hydrogen bonding, van der Waals interaction, metal affinity, biological affinity, and combinations thereof. In certain such embodiments, the adsorption unit used in Step B is a monolith having an anion exchange adsorptive mechanism. In certain such embodiments, wherein the adsorption unit used in Step B is a monolith having an anion exchange adsorptive mechanism provided by quaternary amine moieties. In other such embodiments, the adsorption unit used in Step B is a monolith having a cation exchange adsorptive mechanism. In certain such embodiments, the adsorption unit used in Step B is a monolith having a cation exchange adsorptive mechanism provided by SO3 moieties.

In certain aspects, the preceding embodiments involving Step C or Step E provide a process wherein the adsorption unit used in Step C is a monolith having an anion exchange adsorptive mechanism. In certain such embodiments, the adsorption unit used in Step C is a monolith having an anion exchange adsorptive mechanism provided by quaternary amine moieties. In other such embodiments, the adsorption unit used in Step E is a monolith having an anion exchange adsorptive mechanism; in certain such embodiments, the adsorption unit used in Step E is a monolith having an anion exchange adsorptive mechanism provided by quaternary amine moieties. In other such embodiments, the adsorption unit used in Step C is a monolith having a hydrophobic interaction adsorptive mechanism; in certain such embodiments, the adsorption unit used in Step C is a monolith having a hydrophobic interaction adsorptive mechanism provided by phenyl moieties. In other such embodiments, the adsorption unit used in Step E is a monolith having a hydrophobic interaction adsorptive mechanism; in certain such embodiments, the adsorption unit used in Step E is a monolith having a hydrophobic interaction adsorptive mechanism provided by phenyl moieties.

In certain aspects, the preceding embodiments involving Step C provide a process wherein Step B is performed in a flow through mode and Step C is performed in a bind-elute mode. In certain aspects, the preceding embodiments involving Step E provide a process wherein Step B is performed in a flow through mode and Step E is performed in a bind-elute mode.

In certain aspects, the preceding embodiments provide a process where the biological products have a hydrodynamic diameter between 10 nm and 100 microns. In certain aspects, the preceding embodiments provide a process wherein the biological product is selected from a DNA plasmid, a virus particle, a virus-like particle, a cellular organelle, a cell, an antibody, and a non-antibody protein. In other such embodiments, a source of the preparation is a cell culture harvest or a naturally occurring body fluid selected from the group consisting of serum, plasma, milk, and fluid from a tissue homogenate.

In certain aspects, the preceding embodiments provide a process such that the preparation is subjected to a fractionation or clarification process prior to Step A. In certain such embodiments, the fraction or clarification process prior to Step A reduces the amount of chromatin in the preparation by at least about 50%, 60%, 70%, 80%, 90%, 95% or more than 95%. In certain aspects, the preceding embodiments provide a process such that the preparation is provided in a form having less than 25%, 20%, 15%, 10%, or 5% of the chromatin residing in a source sample from which the preparation was obtained.

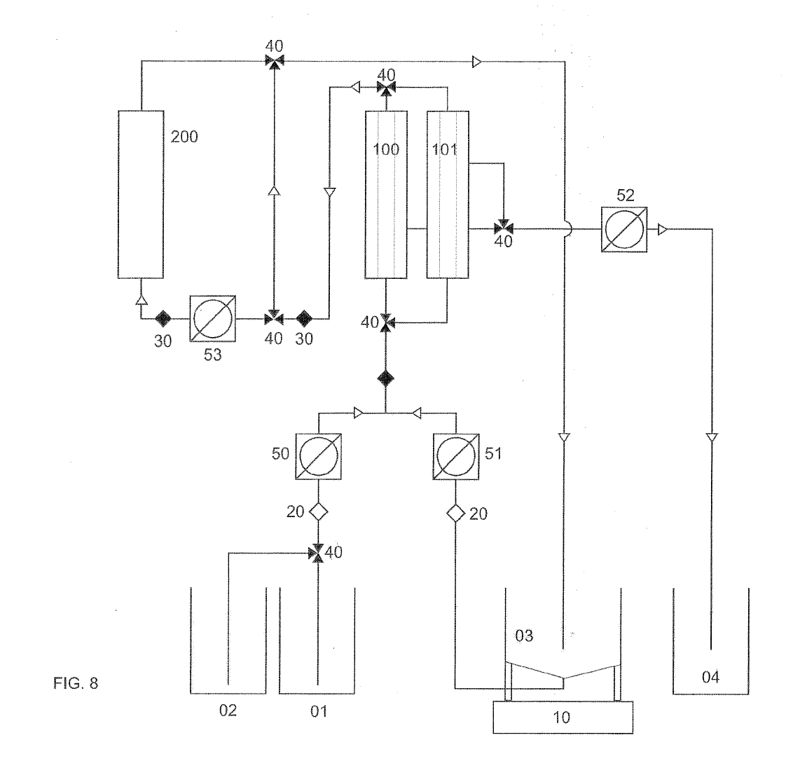

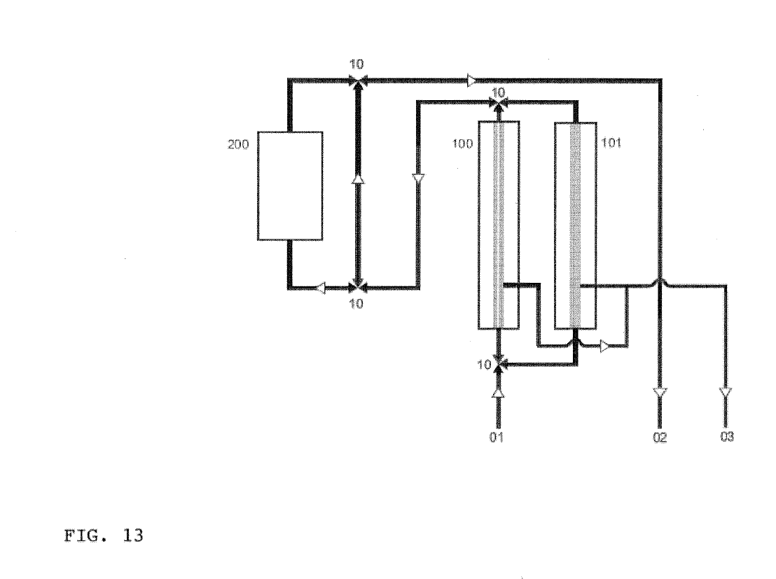

In certain aspects, the preceding embodiments provide a process such that the apparatus has two tangential flow filtration subunits and at least one adsorption unit such that the two tangential flow filtration subunits are plumbed in parallel and either of the two tangential flow filtration subunits can be combined with the adsorption unit in the second configuration. An advantage of such an embodiment is that a purification process can be commenced with one tangential flow filtration subunit and completed using the second tangential flow filtration unit to avoid the effect of any fouling of the first tangential flow filtration subunit upon later stages of the process. Exemplary configurations of such embodiments are illustrated in FIGS. 8 and 13. For example, in certain such embodiments, Step A may be commenced with the first tangential flow filtration subunit and completed (along with all subsequent steps) with the second tangential flow filtration subunit. In other such embodiments, Step A may be performed with the first tangential flow filtration subunit and all subsequent steps (including subsequent performances of Step A) may be completed with the second tangential flow filtration subunit. In yet other such embodiments, Steps A and B may be performed with the first tangential flow filtration subunit and Step C and any subsequent steps (including subsequent performances of Step A) may be completed with the second tangential flow filtration subunit.

In certain aspects, the preceding embodiments of the invention provide processes, preferably multi-step, which are performed according to a format without buffer equilibration prior to performance of a step such that buffer equilibration is performed during the course of some or all of such purification step. Advantages of such embodiments include the more efficient use of buffers and other resources as less volume is required for sample handling and processing.

In certain aspects of the invention concerning apparatus or methods of using such apparatus, the membrane of the tangential flow filtration subunits have a porosity chosen such that at least a minimum percentage of non-adsorbed solutes with a hydrodynamic diameter greater than a selected size are retained on the basis of size, but non-adsorbed solutes with a hydrodynamic diameter less than the selected size are permitted to pass through the membrane. In certain embodiments, the minimum percentage can be any amount between 50% and 100%; in certain such embodiments the minimum percentage is 50%, 60%, 70%, 80%, 90%, 95%, or 99%. In certain of these embodiments such as those directed to purification of an IgG antibody, the selected size may be any amount between about 10 nm and 15 nm; in certain such embodiments the selected size may be approximately 10 nm, 11 nm, 12 nm, 13 nm, 14, nm, or 15 nm.

In certain aspects of the invention concerning apparatus or methods of using such apparatus, the membrane of the tangential flow filtration subunits have a porosity that is characterized as having an average pore size of about 3 nm to about 6 nm, or about 6 nm, or about 5 nm, or about 4 nm, or about 3 nm. In certain aspects of the invention concerning apparatus or methods of using such apparatus, the membrane of the tangential flow filtration subunits have a porosity that is characterized as having a maximum pore size of about 9 nm or less, or about 8 nm or less, or about 7 nm or less, or about 6 nm or less, or about 5 nm or less. The hydrodynamic diameter of IgG antibodies, as measured according to their longest dimension, tend to be approximately 10-15 nm with some variation dependent upon local conditions. Given the flexibility of antibodies and variation in their size, selection of a pore size to retain antibodies typically requires a pore size appreciably smaller than the hydrodynamic diameter of the antibody of interest. For example, a maximum pore size of approximately 9 nm might retain substantially all of the larger IgGs whereas some smaller or more flexible IgGs may require a smaller maximum pore size such as approximately 5 nm. Moreover, pore size in a membrane can be expected to have a distribution such that the average pore size is appreciably smaller than the maximum pore size. For example, a membrane with a maximum pore size of approximately 9 nm may have an average pore size of 6 nm; similarly, a membrane with a maximum pore size of approximately 5 nm may have an average pore size of 3 nm. Skilled practitioners will appreciate how to adapt the foregoing discussion to the selection of membrane porosities suitable for other desired biological products based upon knowledge of their approximate hydrodynamic diameters.

In certain aspects, the invention provides an apparatus for purifying a biological product from a preparation where the apparatus includes (a) multiple purification subunits comprising a first tangential flow filtration subunit equipped with at least one porous membrane having pores with a porosity sufficient to retain practically all of (or at least 50%, 60%, 70%, 80%, 90%, 95% or more than 95% of) the biological product and one or more adsorption subunits; (b) multiple conduits connecting the multiple purification units; (c) valves that direct flow through the multiple conduits and permit isolation of one or more of the multiple purification units from each other; (d) pumps configured to induce flow and control differential pressure within one or more portions of the apparatus; and (e) conduits for supply of the preparation to the apparatus, preferably to the input of the tangential flow filtration unit; wherein the multiple conduits and associated pumps and valves allow cycling of the biological product through the apparatus according to multiple alternate configurations including (i) a first configuration for continuous flow such that the retentate line output of the tangential flow filtration subunit may be collected as recyclate and returned to the input of the tangential flow filtration unit, (ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and, (iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit.

In certain aspects, the preceding embodiments provide an apparatus which also has a second tangential flow filtration purification unit equipped with at least one porous membrane having pores with a porosity sufficient to retain practically all of the biological product, and a second adsorption unit, wherein the multiple conduits additionally allow cycling of the biological product through the apparatus according to multiple alternate configurations including the first configuration, the second configuration, the third configuration and (iv) a fourth configuration for continuous flow such that the retentate line output of the second tangential flow filtration subunit may be collected as recyclate and returned to the input of the second tangential flow filtration unit, and (v) a fifth configuration for continuous flow such that the retentate line output of the second tangential flow filtration connects to the input for the second adsorption subunit such that the output of such adsorption unit may be collected as recyclate and returned to the input of the second tangential flow filtration unit.

In certain aspects, the invention provides an apparatus for purifying a biological product from a preparation where the apparatus includes (a) multiple purification subunits including a first tangential flow filtration subunit equipped with at least one porous membrane having an average pore size from about 2.5 nm to about 5000 nm, and one or more adsorption subunits; (b) multiple conduits connecting the multiple purification units; (c) valves that direct flow through the multiple conduits and permit isolation of one or more of the multiple purification units from each other; (d) pumps configured to induce flow and control differential pressure within one or more portions of the apparatus; and (e) conduits for supply of the preparation to the apparatus, preferably to the input of the tangential flow filtration unit; wherein the multiple conduits and associated pumps and valves allow cycling of the biological product through the apparatus according to multiple alternate configurations including (i) a first configuration for continuous flow such that the retentate line output of the tangential flow filtration subunit may be collected as recyclate and returned to the input of the tangential flow filtration unit, (ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and, (iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit. In certain aspects, the preceding embodiments provide an apparatus which also has a second tangential flow filtration purification unit equipped with at least one porous membrane having an average pore size from about 2.5 nm to about 5000 nm, and a second adsorption unit, wherein the multiple conduits additionally allow cycling of the biological product through the apparatus according to multiple alternate configurations including the first configuration, the second configuration, the third configuration and (iv) a fourth configuration for continuous flow such that the retentate line output of the second tangential flow filtration subunit may be collected as recyclate and returned to the input of the second tangential flow filtration unit, and (v) a fifth configuration for continuous flow such that the retentate line output of the second tangential flow filtration connects to the input for the second adsorption subunit such that the output of such adsorption unit may be collected as recyclate and returned to the input of the second tangential flow filtration unit. In certain such embodiments, the porous membranes of the tangential flow filtration subunits have an average pore size from about 5 nm to about 1000 nm, or from about 10 nm to about 500 nm, or from about 25 nm to about 100 nm. In certain of the preceding embodiments, the porous membranes of the tangential flow filtration subunits have an average pore size selected to retain at least 99% of the desired biological product on the basis of its hydrodynamic radius.

In certain of the preceding embodiments, the porous membranes of the tangential flow filtration subunits have an average pore size selected to be greater than the average hydrodynamic radius of the desired biological product. In certain aspects, the preceding embodiments provide an apparatus such that the membranes of the first and second tangential flow subunits are substantially the same. In other such embodiments, the membranes of the first and second tangential flow subunits have different chemical compositions and porosities that are substantially similar. In still other such embodiments, the membranes of the first and second tangential flow subunits have different chemical compositions and different porosities; In certain such embodiments, one membrane is a polyethersulfone membrane with 0.2 micron pores and the other membrane is a cellulose membrane with pore sizes corresponding with a globular protein with a mass of 30 kDa.

In certain aspects, the preceding embodiments provide an apparatus wherein one or more membranes within one or more of the tangential flow filtration subunits has adsorptive surface characteristics such that it would be suitable for performing adsorption chromatography.

In certain aspects, the preceding embodiments provide an apparatus wherein a physical format of the membrane within the tangential flow filtration subunit is selected from the group consisting of a sheet, a wound (rolled) sheet, a hollow fiber, and combinations thereof. In certain other embodiments, the physical format of the adsorptive subunits is a column packed with adsorptive particles; a monolith, one or more adsorptive membranes, one or more sheets or one or more hollow fibers, or a combination thereof.

In certain aspects, the preceding embodiments provide an apparatus wherein an adsorptive mechanism employed by the adsorptive subunits is independently any of electrostatic interaction, hydrophobic interaction, pi-pi binding, hydrogen bonding, van der Waals interaction, metal affinity, biological affinity, or a combination thereof. In certain such embodiments, the adsorption unit used in the second configuration is a monolith having an anion exchange adsorptive mechanism; in certain such embodiments, the adsorption unit used in the second configuration is a monolith having an anion exchange adsorptive mechanism provided by quaternary amine moieties. In other such embodiments, the adsorption unit used in the second configuration is a monolith having a cation exchange adsorptive mechanism; in certain such embodiments, the adsorption unit used in the second configuration is a monolith having a cation exchange adsorptive mechanism provided by SO3 moieties.

In certain aspects, the preceding embodiments provide an apparatus, wherein the adsorption unit used in the third configuration is a monolith having an anion exchange adsorptive mechanism; in certain such embodiments, the adsorption unit used in the third configuration is a monolith having an anion exchange adsorptive mechanism provided by quaternary amine moieties.

In certain aspects, the preceding embodiments provide an apparatus, wherein the adsorption unit used in the fifth configuration is a monolith having an anion exchange adsorptive mechanism; in certain such embodiments, the adsorption unit used in the fifth configuration is a monolith having an anion exchange adsorptive mechanism provided by quaternary amine moieties.

In certain aspects, the preceding embodiments provide an apparatus wherein the adsorption unit used in the third configuration is a monolith having a hydrophobic interaction adsorptive mechanism; in certain such embodiments, the adsorption unit used in the third configuration is a monolith having a hydrophobic interaction adsorptive mechanism provided by phenyl moieties.

In certain aspects, the preceding embodiments provide an apparatus. wherein the adsorption unit used in the fifth configuration is a monolith having a hydrophobic interaction adsorptive mechanism; in certain such embodiments, the adsorption unit used in the fifth configuration is a monolith having a hydrophobic interaction adsorptive mechanism provided by phenyl moieties.

In certain aspects, the preceding embodiments provide an apparatus which also includes one or more processors configured by computer readable instructions to control the pumps and valves to pass the preparation through a fluid path in accordance with path instructions, wherein the path instructions define a sequence of purification units; in certain such embodiments, the apparatus also includes an interface configured to receive entry or selection by a user of path instructions; in certain such embodiments, the interface is a computer, memory, network, wireless, or combinations thereof.

In certain aspects, the preceding embodiments provide an apparatus which is scaled to perform at multi-milligram scale or at multi-gram scale or at multi-kilogram scale.

In certain aspects, the preceding embodiments provide an apparatus having two tangential flow filtration subunits and at least one adsorption unit such that the two tangential flow filtration subunits are plumbed in parallel and either of the two tangential flow filtration subunits can be combined with the adsorption unit in the second configuration. An advantage of such an embodiment is that a purification process can be commenced with one tangential flow filtration subunit and completed using the second tangential flow filtration unit to avoid the effect of any fouling of the first tangential flow filtration subunit upon later stages of the process. Exemplary configurations of such embodiments are illustrated in FIGS. 8 and 13.

In some embodiments, there are provided apparatuses and methods of using the same, the apparatuses comprising a vessel containing a preparation of a desired biological product; that is integrated with at least one tangential flow filtration subunit with a membrane that retains the desired product from the preparation in a soluble state, and further integrates at least one adsorption subunit, and may optionally include a second or third or additional adsorption subunit; and may optionally include a second tangential flow filtration subunit, equipped with a membrane that retains the product in a precipitated state, or in a state where it is associated with a plurality of particles. Through development and use of the disclosed apparatus and processes, many benefits have been discovered that substantially and surprisingly exceed the capabilities of known apparatus and processes for conducting purification.

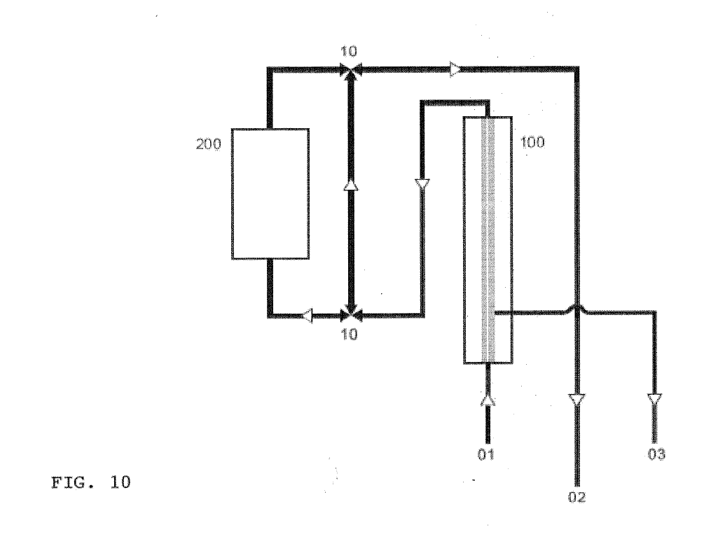

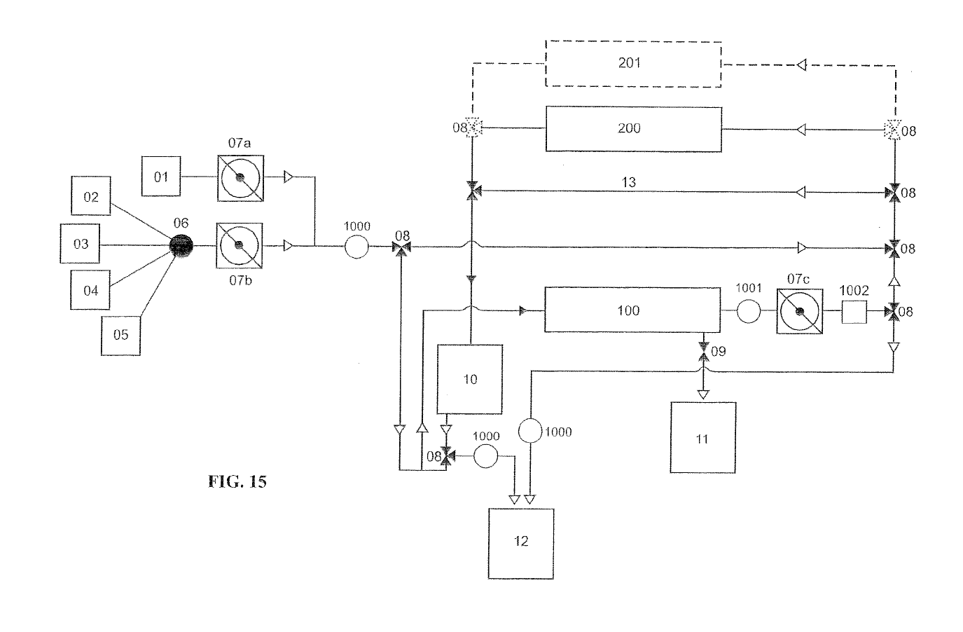

The figures (e.g., FIG. 1, FIG. 10 or FIG. 15) illustrate apparatus configurations for purifying a desired protein, such as an IgG monoclonal antibody, from a clarified cell culture harvest, where the desired protein remains soluble throughout the entire process. The tangential flow subunit (100) is equipped with an inert membrane with a pore size distribution sufficiently small to prevent unacceptable loss of IgG, but large enough to permit the passage of smaller contaminants. In practice, this requirement may be served by a membrane with pores with an average size corresponding to a hypothetical globular protein with a mass of up to about 50 kDa. It is understood that the range of pore sizes in such a membrane includes pores down to half that size or less, and pores up to twice that size or more, generally in a Gaussian distribution around the average. Consequently, it is also understood that a membrane with a larger average pore size, but a narrower range of pore size may perform adequately or better. As a general matter, the membrane with the highest pore size that does not permit unacceptable loss of IgG will be most desirable, so far as the membrane material is inert to sample components. Regenerated cellulose membranes, which are highly inert, may therefore by preferred over polyethersulfone (PES) membranes, which are more hydrophobic and have a higher tendency to participate in nonspecific chemical interactions that may limit contaminant removal and reduce product recovery. The adsorption subunit in the figures (200) may be a fixed bed anion exchange device such as a membrane adsorber, or a monolith, or a column packed with porous particles. As a general matter, the adsorption subunit may be sized relative to the tangential flow filtration subunit so that the adsorption subunit does not restrict the flow rate supported by the tangential flow filtration subunit. As indicated in the figures, the apparatus includes pumps, valves, tubing, and monitoring devices as necessary to channel flow through the apparatus as desired, and optionally a controller to support partial or completely automated operation, if desired.

In one or more embodiments employing the apparatus configurations of the figures, the first step is to concentrate the input protein preparation to reduce the overall process volume. In the case of an IgG monoclonal antibody, the first step may be to concentrate the antibody to 10 g/L, or 25 g/L, or 50 g/L, or 60 g/L, or 70 g/L, or 80 g/L, or 90 g/L, or 100 g/L or a greater, lesser, or intermediate concentration. It will be apparent to persons of skill in the art, that reducing process volume reduces water consumption through the subsequent processing steps, which reduces chemical consumption and processing time by the same increment.

In one or more embodiments employing an apparatus configuration according to one or more of the figures, the second step is to perform virus inactivation of the concentrated antibody. In one such example, buffer is exchanged through the tangential flow filtration subunit (100) from the original buffer in which the antibody was introduced, to a buffer containing, for example, 100 mM acetic acid, 1.7 M NaCl, pH 3.5. Antibody concentration is held relatively constant during this process to maintain the benefits of low processing volume and not requiring extra tankage.

In one or more embodiments employing an apparatus configuration according to one or more of the figures, the third step is to contact the desired protein preparation with the at least one adsorption subunit. In one such example, an anion exchange monolith (200) is put in and the protein preparation, initially buffered with 100 mM acetic acid, 1.7 M NaCl, pH 3.5, is contacted with the monolith. The buffer is exchanged through tangential flow filtration subunit (100) to a buffer comprising 50 mM Tris, pH 8.0. During the buffer exchange, the IgG preparation and the monolith experience conditions ranging from pH 3.5 to pH 8.0, and 1.7 M NaCl to a nominal absence of NaCl. At the point where the IgG preparation reaches 50 mM Tris, pH 8.0, acidic contaminants will have become bound to the anion exchange monolith. At this point, the flow path is changed to a configuration that allows the monolith to be rinsed with clean 50 mM Tris, pH 8.0 buffer, to recover the antibody from the monolith and lines leading to and from it. These steps highlight many unique capabilities of embodiments of certain embodiments of the system, the first of which is illustrated by a conceptual example. The polynucleotide DNA is highly electronegative. The protein lysozyme is highly electropositive. When placed together in a buffer with low or no NaCl, DNA binds to lysozyme and they co-precipitate. If sufficient NaCl is added, they resolubilize and remain independent from each other. If the objective were to remove DNA from the desired product lysozyme by the method of anion exchange chromatography, it would be ideal to apply the sample where the two species were soluble and independent from each other, except that the high salt concentration imposes the restriction of making it impossible for the electropositive charges on the anion exchanger to bind the DNA and thereby remove it from the lysozyme. With the disclosed apparatus and process, it is believed that the DNA and lysozyme would be soluble and independent from each other in 100 mM acetic acid, 1.7 M NaCl, pH 3.5. As the salt concentration began to descend, DNA would be attracted more strongly to the anion exchange monolith than to lysozyme because it embodies millions of times more electropositive groups on its surface than lysozyme. Lysozyme would meanwhile be repelled from the electropositive surface of the anion exchange monolith. The more the salt concentration was diminished, the stronger the attraction of the DNA for the anion exchanger, thus enhancing its effective removal from the lysozyme. It is worth particular note that in the entire field of ion exchange chromatography, all other methods and variations require that the sample be equilibrated to the binding conditions before the sample is contacted with the anion exchanger—except one. The one exception is a mode of anion exchange chromatography called void exclusion mode (Nian et al, J. Chromatography A (2012) 127-132), and even in this mode, sample volume is limited to no greater than about 40% of the volume of a column of packed particles. No known variation of anion exchange chromatography has been previously described that supports the ability of the disclosed method to permit the application of unlimited sample volume, at any pH or salt concentration, without a separate previous step to equilibrate the sample to the necessary binding conditions. The disclosed apparatus and method supports that capability. A second unique feature is that certain embodiments of the system eliminate most of the contaminating proteins that would bind to the anion exchanger, and eventually require high capacity from the anion exchanger to remove a large load of such contaminants, before the protein preparation contacts the anion exchanger under conditions where they could bind. Most such contaminants are eliminated through the pores in the tangential flow filtration subunit during the IgG concentration and virus inactivation stages. This leaves the capacity of the anion exchanger relatively unchallenged, which is an important enabling step to permit the use of typically fast-but-low-capacity convective adsorption chromatography materials like membranes and monoliths. To the extent that some such contaminants may remain in the preparation when the anion exchanger is put into line, the initial high salt concentration prevents them from binding and allows additional opportunity for them to be eliminated through the pores of the tangential flow filtration subunit. A third unique feature of certain embodiments of the invention relates to the fact that the protein preparation is cycled through the anion exchange monolith many times as the buffer is exchanged. Virtually all known methods of fixed-bed adsorption chromatography are based on a single passage of sample through a column, such as an anion exchanger. Without ascribing to any particular theory, it is believed that multiple passes through the anion exchanger support more effective binding of contaminants. A fourth unique feature of certain embodiments of the invention relates to better process control. Samples applied to ion exchangers typically differ slightly in their composition from the buffer the exchanger is equilibrated to, particularly including a slightly different, usually higher, concentration of salt. Chloride ions from a salt such as NaCl displace hydroxide ions from the surface of an anion exchanger and cause the pH of the buffer to rise without control for a duration that is also not controlled. Such phenomena are widely known as pH excursions. pH excursions are eliminated with the disclosed methods because the anion exchanger is equilibrated at the same time to the same conditions in which the IgG resides. A fifth unique feature of certain embodiments of the invention also applies to process control. Most IgG samples applied to anion exchangers have been eluted from a previous protein A affinity chromatography step, or a previous cation exchange chromatography step, or other chromatography step. In either case the IgG preparation contains excess salt. To avoid a separate equipment-requiring buffer exchange step, most processes call for the sample to be equilibrated by dilution and pH titration. This increases sample volume, and since it is performed manually, it is difficult to maintain a high degree of reproducibility over different manufacturing lots. With the disclosed apparatus of certain embodiments, the protein preparation is buffer exchanged to precisely the target conditions for each and every run, thereby enhancing reproducibility. A sixth unique feature of certain embodiments of the invention is the reduction in process volume permitted by the fact that chromatography media equilibration, sample preparation, sample application, and contaminant removal are conducted in a single step, instead of the multiple distinct steps typical of column chromatography. No other system of purification enables this capability. This feature compounds the volume reduction achieved by initial concentration of the desired IgG, and the corresponding reductions in water, chemical consumption, and process time. A seventh unique feature of certain embodiments of the invention is the ability to keep the IgG at a high concentration through every single step of the process, such as 10 g/L, or 20 g/L or 50 g/L, or more. No other system of purification enables this capability, or this combination of capabilities.

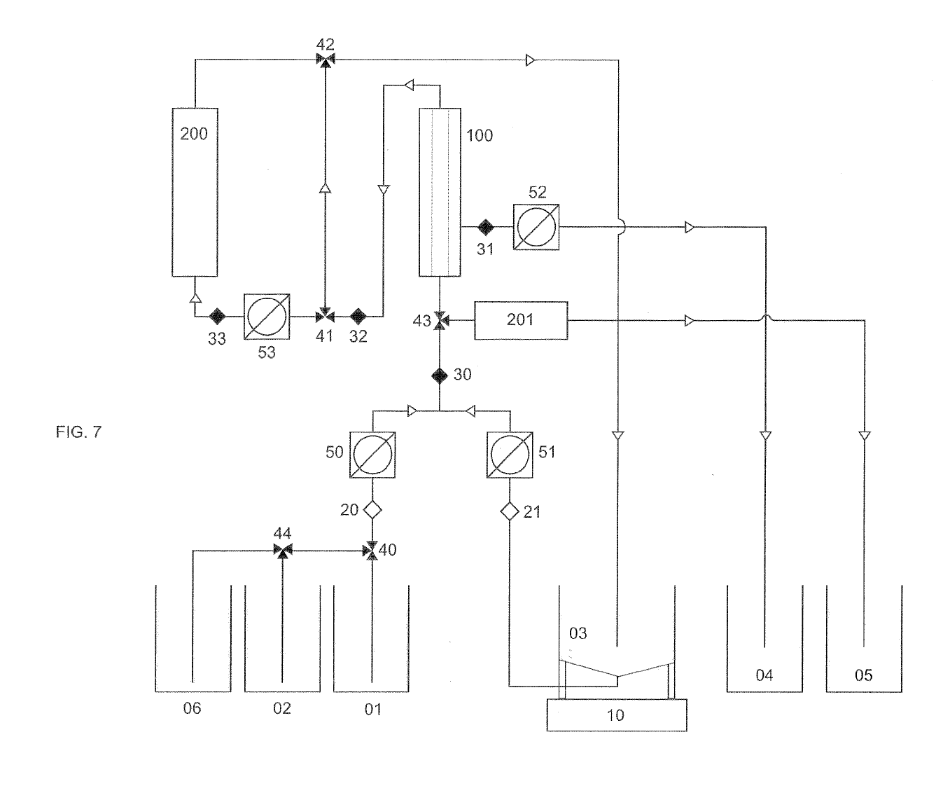

In one or more embodiments employing the apparatus configuration of the figures (e.g., FIG. 1, FIG. 10 or FIG. 15), after the adsorption subunit has been rinsed and put off-line, the IgG preparation is buffer exchanged. A particular benefit of performing this step with the disclosed apparatus and method is that it occurs within the same apparatus as the earlier steps. In some embodiments, the purified IgG preparation may be collected from the system. In other embodiments, the preparation may be further purified by passage through an additional adsorption subunit plumbed to the system where the preparation exits the system as illustrated in FIG. 7. These situations highlight another unique benefit of the disclosed apparatus and methods of certain embodiments of the invention. Normally, losses are incurred at each operational step of a process. These losses sometimes occur because product binds to surfaces within an apparatus, and sometimes because fluids cannot be fully recovered from an apparatus, and the desired antibody residing within the unrecovered volume is lost. Even when recovery is 90% from each step, a 3-step process will suffer a 27% loss (73% recovery) of product. With the present method, an entire process employing multiple fraction mechanisms takes place within a single apparatus that embodies the ability to flush residual desired product from the system. Preliminary data indicate the present system supports more than 90% recovery despite the integrated process involving multiple mechanisms of fractionation. As with all previous steps, product concentration is maintained nearly constant throughout these last steps. This is highly advantageous over traditional chromatography processes where product concentration ranges from high to low values across steps as it is bound and eluted or simply flowed through an adsorption subunit, requiring tankage to accommodate each stage of each step.

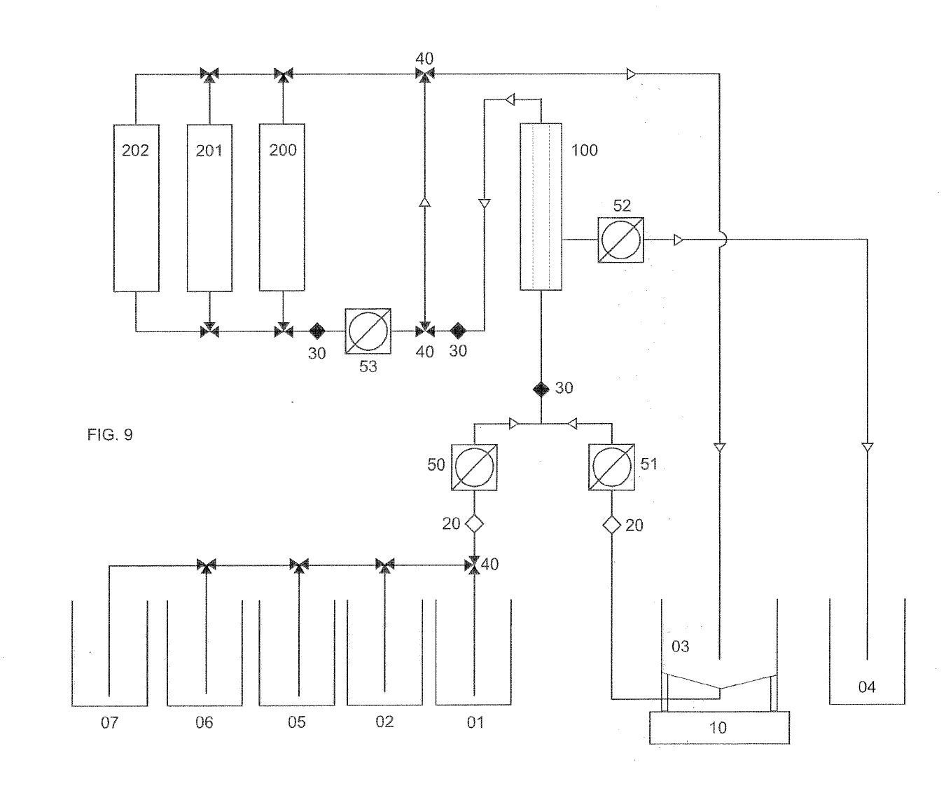

FIG. 9 embodies a more complex configuration for purifying a desired protein, such as an IgG monoclonal antibody, from a clarified cell culture harvest, where the apparatus includes 3 adsorption subunits. Concentration and virus inactivation steps may be performed as described above. Next, the phenyl adsorption membrane subunit is put in line, initially exposed to the IgG preparation in 100 mM acetic acid, 1.7 M NaCl, pH 3.5. With the phenyl membrane subunit still in line, buffer is exchanged through the tangential flow filtration subunit to 50 mM sodium phosphate, 1.7 M NaCl, pH 7.0. After the system reaches these conditions, the phenyl membrane and lines leading to and from it are rinsed to recover the antibody. The anion exchange monolith is put in line and comes into contact with the IgG preparation at 50 mM sodium phosphate, 1.7 M NaCl, pH 7.0. The preparation is then buffer exchanged to 50 mM Tris, pH 8.0. After the IgG preparation reaches these conditions, the anion exchange monolith and lines leading to and from it are rinsed with clean 50 mM Tris, pH 8.0. In some embodiments, the third adsorption subunit may consist of SO3 (cation exchange) monolith, which may be placed in line during or after equilibration to 50 mM Tris, 5 mM NaCl, pH 8. The order of steps may be run in any sequence desired. After completion of the adsorption steps, the preparation may be buffer exchanged into a final or pre-final formulation steps are conducted as described previously. This example highlights another unique feature of the system, which is that the entire process of IgG concentration, virus inactivation, hydrophobic interaction chromatography, anion exchange chromatography, and final formulation requires only 4 buffers, versus IgG capture, intermediate purification, final purification, and formulation that may collectively require more than 10 buffers. This greatly reduces labor associated with buffer preparation, reduces tankage requirements, and reduces the complexity of the process and the apparatus required to conduct it. In another variant embodiment of this configuration, a fourth (external) adsorption subunit may be plumbed to the apparatus.

In some embodiments, the system may be configured with at least one adsorption subunit and at least one tangential flow filtration subunit as described above, and a second adsorption subunit (terminal adsorption subunit) may be plumbed to the outlet of the apparatus in such a way that recirculation including the at least one tangential flow filtration subunit and the terminal adsorption subunit does not occur. In such an embodiment, after rinsing the desired product from the lines with already-used first adsorption subunit, the preparation is buffer exchanged into a buffer suitable for contaminant removal by the terminal adsorption subunit. The terminal adsorption subunit is then put in-line and the desired protein preparation is collected at the outlet as it passes through the terminal adsorption subunit. In a further variant embodiment of this system, the system may be configured with a first and second adsorption subunit in fluid contact, at respective process stages, with the at least one tangential flow filtration subunit, while a third adsorption subunit (terminal adsorption subunit) unit may be plumbed to the outlet of the apparatus in such a way that recirculation including the at least one tangential flow filtration subunit and the terminal adsorption subunit does not occur. In another variant embodiment of this system, more adsorption subunits may be included that are in fluid contact, at respective process stages, with the at least one tangential flow filtration subunit, while a final adsorption subunit (terminal adsorption subunit) may be plumbed to the outlet of the apparatus in such a way that recirculation including the at least one tangential flow filtration subunit and the terminal adsorption subunit does not occur. In any of the above embodiments, the terminal adsorption subunit may be in physical form including a membrane, or a monolith, or a column of packed particles.

Certain embodiments of the invention support another advantage that is enabled by systems such as those exemplified in he figures (e.g., FIG. 1, FIG. 10 or FIG. 15). Since the desired protein is contained within the system by a tangential flow filtration subunit, it is not only tolerant of salts that make retention impossible by the adsorption subunit(s), but also tolerates high concentrations of salts that are particularly known for their ability to dissociate non-specific interactions that maintain the presence of otherwise stable complexes comprising the desired protein and one or more species of contaminants. This permits application of a dissociating wash step in which the desired protein preparation is buffer exchanged into the dissociating buffer, and dissociated contaminants of sufficiently small size are eliminated by their passage through the membrane in the tangential flow filtration subunit. Such a wash step may be performed in place of virus inactivation as described above, or virus inactivation as described above may be considered such a step. Such a wash step may alternatively be performed in addition to a virus inactivation step. In one or more embodiments, a chaotropic salt may include one or more from the group consisting of guanidine hydrochloride, guanidine acetate, sodium thiocyanate, potassium thiocyanate, or another chaotropic salt, where the concentration of one or a combination of chaotropic salts may be 0.05 M, 0.1 M, 0.5 M, 1.0 M, or an intermediate value, or higher so long as the concentration does not lead to permanent denaturation of the desired protein. By the same token, combinations of neutral salts, such as NaCl, or KCl, or K or Na acetate, in combination with other dissociating agents are possible. In one or more such embodiments, a dissociating agent added to a neutral salt may include one or more from the group consisting or urea; sorbitol or xylitol; arginine, lysine, or histidine; a reducing agent such as cysteine, glutathione, or cysteine; a chelating agent such as EDTA, EGTA, citric acid, or TREN; a multivalent cation such as polyethyleneimine, polylysine, or polyarginine, or polyallylamine; an organic solvent such as ethylene glycol, propylene glycol, glycerol, butanol, propanol, ethanol, methanol, or phenoxyethanol; a surfactant such as Tween, Triton, Brij, octaglucoside, CHAPS, or CHAPSO; or a combination of additional dissociating agents. The person of ordinary skill in the art will recognize that there are many ways to applying the principle of dissociating washes as disclosed.

Certain embodiments of the invention produce another advantage that is enabled by the systems such as those exemplified in the figures (e.g., FIG. 1, FIG. 10 or FIG. 15). The technique of tangential flow filtration on membranes with porosity that retains the desired protein is known to result in a reduction of smaller contaminants, but the degree of reduction is limited by the usual practice of minimizing the volume of buffer to the minimum required to reach the targeted buffer specification. During the course of a complete purification with the present apparatus and methods, the larger volume of buffer associated with multiple buffer exchange steps increases the degree of small contaminant reduction through the membrane. This is further enhanced by the diversity of buffer environments the protein preparation experiences, each of which are understood to potentially increase the solubility of different subsets of contaminants, and thereby increase the diversity of contaminants that can be removed though the process as a whole.

In some embodiments, columns packed with porous particles can be employed as adsorption subunits. This may reduce efficiency because their use may require that the concentration of the protein be limited to minimize viscosity, which is understood to hamper diffusive mass transport in particle-based column chromatography systems, and it may require a reduction of volumetric flow rate, however it can be used effectively if desired, for example in situations where membranes or monoliths lacking desired surface chemistries are not available or not economical. In some such embodiments, particle-packed columns can be employed with wide diameters and short bed heights. In conventional chromatography, this typically, and often severely, limits capacity and contaminant removal efficiency, but in the present system, the ability to cycle the preparation multiple times through the column, including in conjunction with buffer exchange-column equilibration, may reduce or overcome that liability and permit a process to benefit from the generally higher capacity of porous particle media in comparison to monoliths and membrane-based adsorptive chromatography devices.

In some embodiments, an adsorption subunit may be in the physical form of a tangential flow filtration membrane and the disclosed apparatus can be used for purification of micro-particulate products such as cells and cellular organelles, as well as for nano-particulate products such as virus particles, or for soluble products such as proteins or polynucleotides, or complex carbohydrates.

In some embodiments, the apparatus can be adapted to proteins larger than IgG. Applying the system to larger proteins provides the opportunity to use tangential flow filtration membranes with larger pore size distributions, if desired, which potentially increases the diversity of contaminant species that can be reduced by passage through the tangential flow filtration subunit as described above.

In some embodiments, the apparatus can be adapted to proteins smaller than IgG. Applying the system to smaller proteins may require the use of membranes with smaller pore size distributions.

In some embodiments, different adsorption subunits employing different surface chemistries may be employed to achieve different selectivities as necessary to achieve the goals of a particular purification process.

In some embodiments, the system can be used to perform adsorption in bind-elute mode, where the desired protein binds to the adsorbent, and is then eluted from the adsorbent, as might be the case where a cation exchange step was applied after IgG concentration, or after virus inactivation. This will also require an additional buffer, which will modestly increase complexity of the system, and increase process volume which will also increase process time, but the integration of a bind-elute mode step within the integrated confines of the apparatus as whole will still deliver dramatic reductions of water volume compared to traditional sequential chromatography operations.

In some of the previous embodiments, fundamentally the same configurations used for purification of proteins can be used for purification of DNA. In some such embodiments, the buffer conditions and the selection of adsorptive surface chemistries may differ, if desired. In one such embodiment, one of the adsorption subunits may be a cation exchanger, where the operating pH is set to about pH 4 to adsorb contaminating proteins. In another such embodiment, one of the convective adsorption subunits may be a hydrophobic interaction medium, operated at a sufficiently high concentration of salt so that proteins are retained while DNA is not.

In some of the previous embodiments, fundamentally the same configurations used for purification of proteins can be used for purification of virus or virus-like particles. In some such embodiments, the buffer conditions and the selection of adsorptive surface chemistries may differ, if desired.

In one or more embodiments, the scale of the apparatus may be miniaturized to support purification of a few mg of a desired product. In one or more embodiments, the scale of the apparatus may be established to support purification of 1-100 mg of a desired product. In one or more embodiments, the scale of the apparatus may be established to support purification of 100 mg to a few grams of a desired product. In one or more embodiments, the scale of the apparatus may be established to support purification of a few to several hundred grams of a desired product. In one or more embodiments, the scale of the apparatus may be established to support purification of several hundred grams to a kilogram of a desired product. In one or more embodiments, the scale of the apparatus may be established to support purification of more than 1 kg of a desired product, or more than 10 kg, or more than 100 kg. In one or more embodiments, the scale of the apparatus may be established to support purification of a smaller, intermediate, or larger amount of a desired product.

In some embodiments, variants of the apparatus and processes of the invention, such as those illustrated in any of FIGS. 1-15, may also be employed to include conduct of an integrated multistep purification process that includes a precipitation step, for example precipitation with a nonionic organic polymer such as polyethylene glycol, or precipitation with a precipitating salt such ammonium sulfate, or sodium citrate, and where the precipitation step is integrated with one adsorption step. Following clarification of a cell culture harvest, the harvest may be concentrated to the desired volume by passage through the tangential flow filtration subunit. Contaminants smaller than the IgG are eliminated by passage through the tangential flow filtration membrane. This step also supports the advantage of allowing the IgG to be concentrated to a specified concentration, which is important for reproducibility of the subsequent precipitation step. The IgG is precipitated by changing the input buffer to one containing a target concentration of a desired precipitating agent at appropriate pH and conditions suitable to adjust the selectivity of the system as desired. In one such embodiment, the IgG-precipitating buffer may be 1.3 sodium citrate, pH 6.0. The apparatus configuration is changed to direct flow through an additional tangential flow filtration subunit equipped with a membrane that retains precipitated protein, but not soluble protein. Unprecipitated contaminants are eliminated through the tangential flow filtration membrane. 1.3 sodium citrate, pH 6.0 continues to be pumped into the system, with the effect of washing the precipitate, and allowing soluble contaminants to continue to pass through the membrane of the tangential flow filtration unit. The apparatus configuration is switched back so that flow is directed through the initial tangential flow filtration subunit. The input buffer is changed to a formulation that cannot maintain the desired product in a precipitated state and thereby achieves its resolubilization, such as 50 mM Tris, pH 8.0. As soon as the antibody is resolubilized, the adsorption subunit, for example consisting of an anion exchange monolith or membrane, is placed in line, and the system is fully buffer exchanged to 50 mM Tris, pH 8.0. The adsorption subunit unit is rinsed with 50 mM Tris, pH 8.0 and taken off line, then the system is optionally buffer exchanged into a preformulation buffer, then the system is rinsed to recover all of the IgG. In a related embodiment, a system including 2 adsorption subunits is employed. In either system configuration, it will be recognized that additional optional steps may integrated, such as virus inactivation and/or dissociation of non-specific interactions in a manner whereby all of the steps are integrated into a single unit operation performed with the one apparatus.

In some embodiments, a variation of the apparatus may be employed that includes a first tangential flow subunit equipped with a membrane that retains the desired product in a soluble state, and a second tangential flow filtration subunit with a membrane that does not retain the desired product, but has the ability to retain suspended particles on which the product may be bound during at least one stage of an integrated multistep process. Such particles, for example such as anion exchange chromatography particles, provide an alternative physical format of a first adsorption subunit. It will be recognized that each ultrafiltration subunit will contain its own filtrate port, and at least one ultrafiltration subunit may additional include a valve on the filtrate port so that waste or product may be directed to different containers, if desired by the operator. The apparatus may optionally include one or more additional adsorption subunit configured as illustrated and described in the examples or with reference to the figures or as otherwise described herein. It will be further recognized that in certain embodiments, the invention provides an apparatus for performing adsorption steps on fluidized particles which may be simplified to omit a fixed-bed adsorption subunit, and omit a tangential flow filtration subunit with a membrane that retains soluble product, leaving an essential core of at least one adsorption subunit consisting of fluidized particles and one tangential flow filtration subunit equipped with a porous membrane that retains the chromatography particles but does not retain soluble species. In certain embodiments, the tangential filtration flow subunits may have porosities sufficient to retain the desired biological product where the size of the porosities is determined with reference to the particles to which the biological product is associated as opposed to the biological product alone.

In any of the embodiments of the invention described herein, the apparatus or process may include monitoring at 2 or more points so that the composition of the materials can be evaluated and documented during processing. Such monitoring may be achieved by UV light absorbance monitors, turbidity monitors, conductivity monitors, pH monitors, pressure monitors, and/or weight monitors, as well as others. In some embodiments, the apparatus may be monitored at more than 2 points. In some such embodiments, a controller may be set to continuously monitor the composition of the liquid flowing through the system at two particular points, and be set to automatically trigger events based on that comparison. In one such example, when the pH and conductivity of the solution exiting a tangential flow filtration subunit matches the conductivity of an input solution, buffer exchange will be judged to have reached completion, and the next event in the sequence of steps will be initiated. In a related example, the event may be initiated when the solution exiting a tangential flow filtration subunit attains 99% identity with the input solution, or 98%, or 97%, or 96%, or 95%, or 90%, or other degree of identity set by the operator. Parameters commonly monitored may include pH, conductivity, and UV absorbance at one or more wavelengths.

In some embodiments, the apparatus will be fitted with capability to remove excess gases from the fluid flow stream, including gases dissolved in the process liquids. In some such embodiments, a device for gas removal may consist of an ordinary bubble trap. In some such embodiments it will be advantageous to place the bubble trap immediately after the pump used to create transmembrane pressure in the system, since outgassing will tend to occur immediately after liquids in the system pass from a pressurized state on the inlet side of the pump, to a relatively non-pressurized state on the outlet side of the pump. In some embodiments, the device for gas removal may consist of a gas exchange membrane fed with a noble gas such as helium or argon, which by virtue of their general immiscibility with water, absorb gases across the membrane from the liquid, but do not themselves enter the liquid flow stream.

In some embodiments one or more of the adsorption subunits is operated in bind-elute mode, where at least one of the operating buffers is formulated to cause the desired product to bind to the one or more adsorption subunits. This approach does not provide many of the advantages of the system when used exclusively in flow-through mode, but may still offer substantially better or more economical performance than traditional chromatography systems.

In some embodiments, the sample is a cell culture harvest, a cell culture supernatant, a protein-containing solution derived from a cell culture, an antibody-containing solution derived from a cell culture, a virus-containing or virus-like particle-containing solution derived from cell culture, or a target species-containing solution from a previous stage of purification. In some embodiments the sample is a naturally occurring fluid containing a product to be fractions, such as serum, plasma, or another bodily fluid, or a tissue homogenate.

It has been unexpectedly discovered, in some embodiments, that the method by which a cell culture is clarified in preparation for purification of a desired proteins substantially affects the ability of the apparatus and methods to obtain the best results.

In one or more of the previous embodiments, the sample includes a clarified cell culture supernatant (CCS). In one such embodiment, the CCS may be clarified by centrifugation, flocculation, filtration, or some combination of these or other techniques. In particular, the ability of a clarification method to achieve at least 90% clearance of chromatin and associated catabolites disproportionately increases the ability of the system to achieve high purification. In some embodiments, the desired product preparation to be processed by the disclosed apparatus and method may be a cell culture harvest that does not contain cells. In one such embodiment, the harvest may be clarified by physical methods such as centrifugation and filtration. In another such embodiment, the harvest may be clarified by contact with one or more positively charged surfaces. In another such embodiment, the harvest may be clarified by a method that removes 90% or more of chromatin and chromatin catabolites. In another such embodiment, the clarification method involves contacting the desired product preparation with soluble and/or insoluble multivalent organic ions.

In one or more of the preceding embodiments, conditioning the protein preparation with organic multivalent ions comprises contacting the sample with an electropositive organic additive. In some such embodiments, the electropositive organic additive comprises at least one species from the group consisting of ethacridine, methylene blue, cetyl trimethylammonium bromide. In some such embodiments, the concentration of such a species, or aggregate concentration of a combination of species is in the range of 0.001 to 1%, or 0.01 to 0.1%, or 0.02 to 0.05%. In some such embodiments the pH of the preparation may be adjusted up to an alkaline value that does not cause significant reduction of recovery of the desired protein. In one such embodiment where the desired protein is an IgG monoclonal antibody, the pH may be adjusted up to a pH value a half pH unit of the antibody isoelectric point, or more if experimental results indicate that antibody recovery is acceptable, but such adjustments are generally not necessary. To the extent that any pH adjustment is made, a value within 1 pH unit of the protein isoelectric point will suffice, or within 1.5 pH units, and in some cases within 2 pH units or more.

In one or more of the preceding embodiments, conditioning the protein preparation with organic multivalent ions comprises contacting the sample with an electronegative organic additive. In some such embodiments, the electronegative organic additive comprises at least one species from the group consisting of heptanoic acid, heptenoic acid, octanoic acid, octenoic acid, nonanoic acid, nonenoic acid, decanoic acid, methyl blue. In some such embodiments, the concentration of such a species, or total concentration of a combination of species is in the range of 0.001 to 10%, or 0.01 to 1%, or 0.1 to 0.5%. In some such embodiments the pH of the preparation may be adjusted down to an acidic value that does not cause significant reduction of recovery of the desired protein. In some such embodiments, the pH of the preparation may be adjusted to the range of 3.5 to 6.5, 4.0 to 6.0, 4.5 to 5.5, 5.0 to 5.3, 5.15 to 5.25, or 5.2, or another intermediate value.

In one or more of the preceding embodiments, conditioning the protein preparation with organic multivalent ions comprises contacting the sample with undissolved allantoin. In some such embodiments, the added allantoin resident in a protein preparation may amount to about 0.6% to 50%, or 0.7 to 20%, or 0.8 to 10%, or 0.9 to 5%, or 1 to 2%, or an intermediate value. In one or more of the preceding embodiments, the average particle size of the dry allantoin is selected to be the smallest size available, with the goal of achieving the highest total surface area of the undissolved allantoin in a supersaturated solution. In one such embodiment, the allantoin is granulated to produce a smaller particle size.

In one or more of the preceding embodiments, conditioning the protein preparation with organic multivalent ions comprises contacting the sample with a nonionic or zwitterionic surfactant at a concentration lower than its critical micelle concentration.

In one or more of the preceding embodiments, conditioning of the protein preparation with organic multivalent ions comprises (i) providing a first component which is a first solid substrate having an electronegative surface; (ii) contacting the protein preparation with the first component, wherein the operating conditions substantially prevent the binding of the desired protein to the first component; and (iii) separating the desired protein with a reduced chromatin content from the first component. In some such embodiments, the first electronegative surface may be accompanied by a second electronegative surface.

In one or more of the preceding embodiments, conditioning of the protein preparation with organic multivalent ions comprises (i) providing a first component which is a first solid substrate having an electropositive surface; (ii) contacting the protein preparation with the first component, wherein the operating conditions substantially prevent the binding of the desired protein to the first component; and (iii) separating the desired protein with a reduced chromatin content from the first component. In some such embodiments, the first electropositive surface bears residues of Tris(2-aminoethyl)amine (TREN). In some such embodiments, the first electropositive surface bears a derivative of TREN, such as a TREN dendrimer created by synthetically creating multiple layers of TREN on the surface, or such as TREN with hydrophobic residues bonded to its amino acid residues. In some such embodiments, the hydrophobic residues may be of an alkyl or aryl composition, or of a combined composition. In some such embodiments, alkyl residues may consist of 3 or 5 or 6 or 7 or 8 or 9 or 10 carbon atoms. In some embodiments, the range of carbon atoms is from 4 to 8. In some such embodiments, the first electropositive surface may be accompanied by a second electropositive surface.

In one or more embodiments, an electropositive solid phase used to condition the preparation employs an amino-based metal chelating ligand such as TREN (Tris(2-aminoethyl)amine), or one of its derivatives or another electropositive chelating ligand, where the ligand is loaded with iron and excess iron is removed before the solid phase is employed to condition the preparation.

In one or more of the preceding embodiments, conditioning of the protein preparation with organic multivalent ions comprises (i) providing a first component which is a first solid substrate having an electropositive surface; (ii) providing a second component which is a second solid substrate having an electronegative surface; (iii) contacting the protein preparation with the first and second components, wherein the first and second components are configured such that the protein preparation may contact both components simultaneously, wherein the operating conditions substantially prevent the binding of the desired protein to the first or second components; and (iv) separating the desired protein with a reduced chromatin content from the first and second components. In some such embodiments, the first electropositive surface bears residues of Tris(2-aminoethyl)amine.

In one or more of the preceding embodiments, conditioning of the protein preparation with organic multivalent ions comprises (i) contacting the protein preparation with at least one solid surface comprising at least one surface-bound ligand capable of binding a metal, wherein the surface-bound ligand capable of binding a metal is initially substantially devoid of a metal, wherein operating conditions are selected to substantially prevent the binding of the desired protein to the at least one solid surface and (ii) separating the protein preparation from the at least one surface-bound ligand.

In one or more of the preceding embodiments, a protein preparation already treated with a soluble electropositive or electronegative organic additive and/or a solid surface bearing an electronegative, electropositive, or metal affinity ligand, may be subsequently flowed through a device, the fluid-contact surface of which comprises positive charges.

In one embodiment illustrating application of a chromatin-directed clarification method, allantoin is added to a cell culture harvest in an amount of 1% (v/v). The cell culture may contain cells, or the cells may previously have been removed. Methylene blue is added to a concentration of 0.025% (w/v). Alternatively, ethacridine may be added to a concentration of 0.025%. Alternatively, 0.025% cetyltrimethyl ammonium bromide may be added to a concentration of 0.025%. Alternatively, a combination of these or other electropositive organic additives may be used at an combined concentration of 0.025%. The mixture is then incubated stiffing for 2 hours. Particles bearing the electropositive metal affinity ligand Tris(2-aminoethyl)amine (TREN) are added in an amount of 2-5% v:v. The mixture is incubated stirring for 4 hours then the solids are removed by any expedient means. The remaining solution containing the desired protein may be optionally flowed through a depth filter bearing positive charges on its fluid contact surface. In some embodiments, the TREN is chemically modified so that its terminal amino acid residues bear hydrophobic residues, where the hydrophobic residues may be aryl, alkyl, or of mixed composition, and if alkyl, with a number of carbon atoms from 1 up to 10.

In another embodiment illustrating application of a chromatin-directed clarification method, allantoin is added to a cell culture harvest in an amount of about 1% (v/v). The cell culture may contain cells, or the cells may previously have been removed. About 0.6% heptanoic acid is added. Alternatively about 0.4% octanoic acid is added, or about 0.4% octenoic acid. Alternatively about 0.3% pelargonic (nonanoic) acid is added, or about 0.4% nonenoic acid. Alternatively about 0.2% capric acid is added. Alternatively, about 0.5% methyl blue is added. Alternatively, a combination of these or other electronegative organic additives may be used. The mixture is then incubated stiffing for 2 hours. Particles bearing the electropositive metal affinity ligand Tris(2-aminoethyl)amine (TREN) are added in an amount of about 2 to about 5% v:v. The mixture is incubated mixing for 4 hours then the solids removed by any expedient method. The remaining solution containing the desired protein may be optionally flowed through a depth filter bearing positive charges on its fluid contact surface. In some embodiments, the TREN is chemically modified so that its terminal amino acid residues bear hydrophobic residues, where the hydrophobic residues may be aryl, alkyl, or of mixed composition, and if alkyl, with a number of carbon atoms from 1 up to 10.

In one or more of the previous embodiments, salt may be added to a clarification mixture to prevent loss of the desired protein through excessive interactions with a soluble or insoluble multivalent organic ion. In some such embodiments, NaCl may be added to increase conductivity to a level corresponding to about 200 mM, with a rough conductivity equivalent of about 20 mS/cm, for the purpose of preventing an IgM antibody or non-antibody protein from binding to components of a chromatin-directed clarification system. In other such embodiments, the NaCl concentration may be elevated to a greater or lesser degree to accommodate a particular recombinant protein. Appropriate salt concentrations for accommodating any particular protein can be quickly and easily estimated by applying a sample of the desired protein to a cation exchanger or an anion exchanger, eluting them with an increasing salt gradient, determining the conductivity at the center of the desired protein peak, then using that conductivity value for the clarification process.

In some embodiments, the desired biological product comprises one selected from the group consisting of a protein, an antibody, a clotting factor, a cellular organelle, a virus, a virus-like particle, a gene therapy vector, a polynucleotide, a cell.

In some embodiments, the desired biological product is a polyclonal or monoclonal antibody of the class IgA, IgD, IgE, IgG, or IgM, or a compound recombinant construct thereof, such as an Fc-fusion protein; or a synthetic compound construct thereof, such as a conjugate.

In some embodiments, the desired biological product is a cellular organelle, such as chloroplast, a mitochondrion, a ribosomes, an exosome, or other organelle.

In some embodiments, the desired biological product is a prokaryotic cell, a eukaryotic cell, a stem cell, or a virus particle.

In one or more of the preceding embodiments, an agent employed to precipitate a protein or virus may be one selected from the group of polyethylene glycol, polypropylene glycol, polyvinylpyrrolidone, dextran, starch, cellulose.

In one or more of the preceding embodiments, an adsorption subunit may be a membrane adsorber, where the term membrane adsorber is understood to represent the common name referring to a microporous membrane, the surface of which has been chemically modified to permit that surface to participate in chemical interactions with species dissolved or suspended in a protein preparation. Membrane adsorbers are commonly formatted with multiple layers of membrane material, for example 15 or more, or fewer. The nature of the chemical modification may be of such a nature to leave the membrane able to participate in one or more from the group consisting of electrostatic interactions, hydrophobic interactions, hydrogen bonding interactions, metal affinity interactions. Among electrostatic interactions, the surface of the membrane may be electropositive, or electronegative, or zwitterionic.

In one or more of the preceding embodiments, an adsorption subunit may be a monolith, the surface of which has been chemically modified to permit that surface to participate in chemical interactions with species dissolved or suspended in a protein preparation. The nature of the chemical modification may be of such a nature to leave the monolith able to participate in one or more from the group consisting of electrostatic interactions, hydrophobic interactions, hydrogen bonding interactions, metal affinity interactions. Among electrostatic interactions, the surface of the membrane may be electropositive, or electronegative, or zwitterionic.

In one or more of the preceding embodiments, an adsorption subunit may be a column of packed particles, the surfaces of which have been chemically modified to permit those surfaces to participate in chemical interactions with species dissolved or suspended in a protein preparation. The nature of the chemical modification may be of such a nature to leave the membrane able to participate in one or more from the group consisting of electrostatic interactions, hydrophobic interactions, hydrogen bonding interactions, metal affinity interactions. Among electrostatic interactions, the surface of the membrane may be electropositive, or electronegative, or zwitterionic.

In one or more of the preceding embodiments, an adsorption subunit may embody a hybrid or compound structure, the surface of which has been chemically modified to permit that surface to participate in chemical interactions with species dissolved or suspended in a protein preparation. In one such embodiment, a hydrid or compound adsorption subunit may comprise structural skeleton upon which a hydrogelatinous phase is presented The nature of the chemical modification may be of such a nature to leave the monolith able to participate in one or more from the group consisting of electrostatic interactions, hydrophobic interactions, hydrogen bonding interactions, metal affinity interactions. Among electrostatic interactions, the surface of the membrane may be electropositive, or electronegative, or zwitterionic.

In one or more of the preceding embodiments, the fluid contact surface of an adsorption subunit is electropositive.

In one or more of the preceding embodiments, an electropositive adsorption subunit is an anion exchanger.

In one or more of the preceding embodiments, an electropositive chromatography subunit embodies additional chemical functionalities, such as the ability to participate in one or more from the group consisting of hydrogen bonds, hydrophobic interactions, pi-pi bonding, metal affinity. In one or more of such embodiments, chromatography media that embody multiple chemical interactivities are referred to as mixed mode, or multimodal media.

In one or more of the preceding embodiments, the fluid contact surface of an adsorption subunit is electronegative.

In one or more of the preceding embodiments, an electronegative adsorption subunit is a cation exchanger.

In one or more of the preceding embodiments, an electronegative adsorption subunit embodies additional chemical functionalities, such as the ability to participate in one or more from the group consisting of hydrogen bonds, hydrophobic interactions, pi-pi bonding, metal affinity. In one or more of such embodiments, chromatography media that embody multiple chemical interactivities are referred to as mixed mode, or multimodal media.

In one or more of the preceding embodiments, the fluid contact surface of an adsorption subunit is hydrophobic. In one such embodiment, the hydrophobicity is conferred by an alkyl chemical moiety, such as a propyl group, a butyl group, a pentyl group, a hexyl group, a heptyl group, an octyl group. In another such embodiment, the hydrophobicity may be conferred by an aryl or aromatic moiety, such as a phenyl group, a benzyl group, a bi-phenyl group.

In one or more of the preceding embodiments, an electronegative adsorption subunit is a hydrophobic interaction chromatography medium.

In one or more of the preceding embodiments, a hydrophobic chromatography subunit embodies additional chemical functionalities, such as the ability to participate in one or more from the group consisting of hydrogen bonds, electrostatic interactions, pi-pi bonding, metal affinity. In one or more of such embodiments, chromatography media that embody multiple chemical interactivities are referred to as mixed mode, or multimodal media.

In one or more of the preceding embodiments, any combination of sample, reagent, and reaction mixture contact surfaces of the apparatus comprise or are lined with a material that is easily removable and optionally disposable

In one or more of the preceding embodiments, a plurality of the modular purification components that come into contact with any combination of the fractionating agents, the biological preparation, reagents, and reaction mixture are disposable; the purification module components comprising one or more of tubing sets, membranes, mixers, and valve-bodies.

In one or more of the preceding embodiments, the system is partially or fully automated.

In one or more of the preceding embodiments, the desired biological product is a protein. In one such embodiment, the protein is an antibody.

In one or more of the previous embodiments, the antibody is an IgG. In one such embodiment, the antibody is a monoclonal IgG.

In one or more of the previous embodiments, the method is used to purify a non-IgG antibody. In one such embodiment the antibody is an IgM.

In one or more of the previous embodiments, the method is used to purify a non-antibody protein. In one such embodiment the non-antibody protein is a clotting protein. In one such embodiment the non-antibody protein is Factor VIII.

In one or more of the previous embodiments, the desired protein is partially purified before being processed by the methods disclosed herein.

In one or more of the previous embodiments, the desired product is a polynucleotide. In one such embodiment, the polynucleotide is DNA. In one such embodiment, the DNA is a plasmid for gene therapy. In a related embodiment, the desired product is RNA.

In one or more of the previous embodiments, the desired product is a virus particle or virus-like particle. In one such embodiment, the virus is used to support the testing of antiviral treatments. In another such embodiment, the virus is used as a vaccine. In another such embodiment, the virus particle is used for antibiotic replacement. In another such embodiment, the virus particle is used as a gene therapy vector.

In one or more of the previous embodiments, a stage of the method may additionally include agents intended to inactivate virus, such as tri(n)butyl phosphate, octanoic acid, methylene blue, ethacridine, chlorhexidine, benzalkonium chloride, or related compounds potentially in combination with other agents such as named above, particularly including organic solvents and surfactants.

In one or more of the previous embodiments, the operating pH may be in the range of 4 to 9, or 5 to 8, or 6 to 7; or 7 to 8, or 5, or 6, or 7, or 8; or an intermediate value.

In some embodiments, there are provided processes comprising providing an apparatus comprising (1) a vessel configured to contain a biological product in a target volume (2) multiple purification units comprising (a) at least one purification unit comprising at least one tangential flow filtration subunit, the at least one tangential flow filtration subunit being equipped with one or more membranes that retain the biological product, while allowing the passage of contaminants having a hydrodynamic radius less than about 50% of the hydrodynamic radius of the biological product, and (b) at least one purification unit comprising at least one adsorption subunit, (3) multiple conduits connecting the vessel and the multiple purification units, thereby allowing cycling through the apparatus, (4) valves that direct flow through the conduits and permit isolation of any one or more of the multiple purification units from each other and the vessel, and (5) pumps configured to induce flow and control differential pressure within one or more portions of the apparatus, the process comprising contacting the preparation one or more times with the at least one tangential flow filtration subunit and the at least one adsorption subunit, when the at least one tangential flow filtration subunit and the at least one adsorption subunit are configured, via the valves, to be in fluid communication with each other, wherein at least one contacting step is performed under conditions that substantially prevent binding of contaminants and the biological product to the least one adsorption subunit, and then altering conditions through the at least one tangential flow filtration subunit to (1) allow binding of contaminants but not the biological product to at least one adsorption subunit, or (2) allow binding of contaminants and the biological product to at least one adsorption subunit followed by altering conditions to release the biological product from the at least one adsorption subunit while retaining at least a portion of contaminants. In some embodiments, when the contacting step is performed more than once, altered condition (1) is selected.

In some embodiments, the at least one tangential flow filtration subunit being equipped with one or more membranes may retain at least 50% of the biological product.

In some embodiments, allowing the passage of contaminants having a hydrodynamic radius less than about 50% of the hydrodynamic radius of the biological product may include a hydrodynamic radius less than about 40%, 30%, 20%, 10%, 5%, or 1% of the hydrodynamic radius of the biological product.

In the practice of the processes disclosed herein, one skilled in the art will recognize that the exact “conditions” employed in the processes disclosed herein depend on numerous variables, depending on, inter alia, the selection of composition for the adsorption subunit media and the biological target. The skilled artisan will equally appreciate that the processes of customizing the conditions to a particular product follow the same familiar principles and are fundamentally the same as they are for customizing conditions for traditional methods. Thus, operators may obtain the benefits of the disclosed apparatus and methods with the same skills they have developed to develop purification methods with traditional adsorption chromatography methods. By way of example only, a biological product may be an IgG antibody and the at least one adsorption subunit may be a cation exchanger, and the “conditions” may comprise a concentration of salt sufficiently high to prevent the binding of the IgG antibody and contaminants. Thus, a possible condition under the circumstance may be greater than 0.1 M NaCl. The skilled artisan will appreciate this dependence on both the media and the biological product to select appropriate conditions. Thus, it will be understood that it is a matter of routine practice to select appropriate operational conditions when employing anion exchangers, performing hydrophobic interaction chromatography, steric exclusion, hydroxyapatite, and any number of known multimodal chromatography techniques.

In some embodiments, processes disclosed herein may further comprise contacting the preparation one or more times with the at least one tangential flow filtration subunit while bypassing the at least one adsorption subunit and optionally contacting the preparation with one or more further tangential flow filtration subunits, each independently from other purification units.

In some embodiments, the at least one adsorption subunit is an ion exchanger and the conditions comprise a neutral salt at a concentration greater than necessary to prevent binding of contaminants by an increment greater than about 0.05M up to greater than about 2 M. In some embodiments, the conditions may comprise up to a concentration corresponding to saturated salt.

In some embodiments, the at least one adsorption subunit may be a hydrophobic interaction chromatography medium and the condition may comprise a salt at a concentration less than necessary for some contaminants to bind, by an increment greater than about 0.05M up to about greater than 2 M. In some embodiments a zero concentration of the salt may be employed.

In some embodiments, the apparatus may comprise a single tangential flow filtration subunit and two adsorption subunits, wherein one of the two adsorption subunits is an anion exchanger, and the other is a hydrophobic interaction chromatography medium.

In some embodiments, the apparatus may comprise a single tangential flow filtration subunit, one at least one adsorption subunit, and one additional adsorption subunit, wherein the at least one adsorption subunits is an anion exchanger, and the one additional adsorption subunit is a hydrophobic interaction chromatography medium.

In some embodiments, the apparatus may comprise a single tangential flow filtration subunit, one at least one adsorption subunit, and one additional adsorption subunit, wherein the at least one adsorption subunits is an anion exchanger, and an additional adsorption subunit is a hydrophobic interaction chromatography medium, where the hydrophobic interaction chromatography subunit is plumbed in such a way as to be in recirculating fluid contact with the at least one tangential flow filtration subunit during at least one stage of the process; and the anion exchange adsorption subunit (terminal chromatography subunit) is plumbed at or proximal to the outlet of the apparatus in such a way that the recirculating fluid contact between the terminal adsorption subunit and the at least one adsorption subunit do not occur, and the protein preparation is processed through the terminal adsorption subunit as it exits the apparatus for collection.

In some embodiments, the apparatus may comprise a single tangential flow filtration subunit, one at least one adsorption subunit, and one additional adsorption subunit, i.e., at least two adsorption subunits, wherein at least one adsorption subunits is an cation exchanger, and the additional adsorption subunit is a hydrophobic interaction chromatography medium, both plumbed in such a way as to permit each, respectively, to be in recirculating fluid contact with the at least one tangential flow filtration subunit during at least one stage of the process; and a third adsorption subunit (terminal chromatography subunit) in the form of a chromatography medium with an electropositive surface, plumbed at or proximal to the outlet of the apparatus in such a way that the recirculating fluid contact between the terminal adsorption subunit and the at least one adsorption subunit do not occur, and the protein preparation is processed through the terminal adsorption subunit as it exits the apparatus for collection.

In some embodiments, the at least one adsorption subunit may comprise an electropositive chromatography medium combining positive charges with an excess of hydrogen bonding residues, an excess of hydrophobic residues, or both. In some embodiments, an exemplary electropositive chromatography medium is Capto adhere.

In some embodiments, the apparatus may comprise two tangential flow filtration subunits, one of the two tangential flow filtration subunits retains the biological product adsorbed onto fluidized particles and one of the two tangential flow filtration subunits retains the biological product in a soluble state, the apparatus further comprising at least one adsorption subunit that is an anion exchanger.

In some embodiments, the apparatus may comprise two tangential flow filtration subunits, one of the two tangential flow filtration subunits retains the biological product in a precipitated state and one of the two tangential flow filtration subunits retains the biological product in a soluble state, the apparatus further comprising at least one adsorption subunit that is an anion exchanger.

In some embodiments, an adsorption subunit comprises one or more membranes, one or more monoliths, or particles in a fixed bed or fluidized bed format.

In some embodiments, the biological product is selected from the group consisting of a DNA plasmid, a virus particle, a protein, a recombinant protein, an antibody, a monoclonal antibody, an IgG, an IgM, a non-antibody protein, a clotting factor, Factor VIII, Factor VIII-von Willebrand complex, and fibrinogen.

In some embodiments, the preparation may comprise a naturally occurring body fluid selected from the group consisting of serum, plasma, milk, fluid from a tissue homogenate, and cell culture harvest, and wherein the preparation has been processed to remove physical debris.

In some embodiments, the preparation may have been alternatively or further processed to remove the majority of chromatin.

In some embodiments, the preparation has been processed to remove the majority of chromatin by contact with at least one organic additive selected from the group consisting of a ureide, and electropositive ion, and electronegative ion, an organic solvent, an organic polymer, a surfactant. In some such embodiments, and electropositive ion is soluble. In some such embodiments an electropositive ion is not soluble, by virtue of being covalently attached to a solid surface. In some such embodiments, and electronegative ion is soluble. In some such embodiments an electronegative ion is not soluble, by virtue of being covalently attached to a solid surface.

In some embodiments, the biological product is a recombinant protein in a cell culture harvest, the process comprising conditioning cell culture harvest to remove 90% or more (or 95% or more) of chromatin from the preparation prior to the contacting steps.

In some embodiments, there are provided apparatuses for purifying biological product from a preparation comprising (1) a vessel configured to contain the biological product in a target volume (2) multiple purification units comprising (a) at least one purification unit comprising at least one tangential flow filtration subunit, the at least one tangential flow filtration subunit being equipped with one or more membranes that retain the biological product, while allowing the passage of contaminants having a hydrodynamic radius less than about 50% of the hydrodynamic radius of the biological product, and (b) at least one purification unit comprising at least one adsorption subunit, (3) multiple conduits connecting the vessel and the multiple purification units, thereby allowing cycling through the apparatus, (4) valves that direct flow through the conduits and permit isolation of any one or more of the multiple purification units from each other and the vessel, and (5) pumps configured to induce flow and control differential pressure within one or more portions of the apparatus.

In some embodiments, there are at least two tangential flow filtration subunits, wherein a first tangential flow filtration unit comprises a first membrane having a porosity selected to retain the biological product in a soluble state and wherein the second tangential flow filtration subunit comprises a second membrane having a porosity selected to retain the biological product only when the biological product is in a precipitated or particle-associated state.

In some embodiments, the vessel further comprises a mixer.

In some embodiments, the tangential flow filtration subunit comprises a membrane having a porosity selected to retain the biological product.

In some embodiments, a surface of the adsorption subunit possesses a functional moiety capable of an interaction selected from the group consisting of electrostatic interactions, hydrophobic interactions, pi-pi bonding, hydrogen bonding, metal affinity, biological affinity, and combinations thereof.

In some embodiments, the apparatus comprises two adsorption subunits. In some such embodiments, the surfaces of the two adsorption subunits possess different functional moieties capable of an interaction selected from the group consisting of electrostatic interactions, hydrophobic interactions, pi-pi bonding, hydrogen bonding, metal affinity, biological affinity, and combinations thereof.

In some embodiments, the apparatus comprises three or more adsorption subunits. In some such embodiments, the surfaces of the respective adsorption subunits possess different functional moieties capable of an interaction selected from the group consisting of electrostatic interactions, hydrophobic interactions, pi-pi bonding, hydrogen bonding, metal affinity, biological affinity, and combinations thereof.

In some embodiments, the apparatus may further comprise one or more processors configured by computer readable instructions to control the pumps and valves to pass the preparation through a fluid path in accordance with path instructions, wherein the path instructions define a sequence of purification units. In some such embodiments, the path instructions specify the vessel as the end of the path.

In some embodiments, the apparatus may further comprise an interface configured to receive entry or selection by a user of path instructions. In some such embodiments, the interface is a computer, memory, network, wireless, or combinations thereof.

In some embodiments, the apparatus is scaled to perform at multi-milligram scale. In some embodiments, the apparatus is scaled to perform at multi-gram scale. In some embodiments, the apparatus is scaled to perform at multi-kilogram scale.

In some aspects, embodiments disclosed herein relate to processes comprising providing an apparatus comprising a vessel configured to contain a biological product in a target volume, multiple purification units comprising at least one purification unit comprising at least one tangential flow filtration subunit, the at least one tangential flow filtration subunit being equipped with one or more membranes that retain the biological product, while allowing the passage of contaminants having a hydrodynamic radius less than about 50% of the hydrodynamic radius of the biological product, and at least one purification unit comprising at least one adsorption subunit, multiple conduits connecting the vessel and the multiple purification units, thereby allowing cycling through the apparatus, valves that direct flow through the conduits and permit isolation of any one or more of the multiple purification units from each other and the vessel, and pumps configured to induce flow and control differential pressure within one or more portions of the apparatus, the process comprising contacting the preparation one or more times with the at least one tangential flow filtration subunit and the at least one adsorption subunit, when the at least one tangential flow filtration subunit and the at least one adsorption subunit are configured, via the valves, to be in fluid communication with each other, wherein at least one contacting step is performed under conditions that substantially prevent binding of contaminants and the biological product to the least one adsorption subunit; and then altering conditions through the at least one tangential flow filtration subunit to: (1) allow binding of contaminants but not the biological product to at least one adsorption subunit; or (2) allow binding of contaminants and the biological product to at least one adsorption subunit followed by altering conditions to release the biological product from the at least one adsorption subunit while retaining at least a portion of contaminants.

In some aspects, embodiments disclosed herein relate to apparatuses for purifying biological product from a preparation comprising a vessel configured to contain the biological product in a target volume, multiple purification units comprising at least one purification unit comprising at least one tangential flow filtration subunit, the at least one tangential flow filtration subunit being equipped with one or more membranes that retain the biological product, while allowing the passage of contaminants having a hydrodynamic radius less than about 50% of the hydrodynamic radius of the biological product, and at least one purification unit comprising at least one adsorption subunit, multiple conduits connecting the vessel and the multiple purification units, thereby allowing cycling through the apparatus, valves that direct flow through the conduits and permit isolation of any one or more of the multiple purification units from each other and the vessel, and pumps configured to induce flow and control differential pressure within one or more portions of the apparatus.

Terms are defined so that the embodiments may be understood more readily. Additional definitions are set forth throughout the detailed description.

“Protein” refers to any of a group of complex organic macromolecules that contain carbon, hydrogen, oxygen, nitrogen, and usually sulfur and are composed principally of one or more chains of amino acids linked by peptide bounds. The protein may be of natural or recombinant origin. Proteins may be modified with non-amino acid moieties such as through glycosylation, pegylation, or conjugation with other chemical moieties. Examples of proteins include but are not limited to antibodies, clotting factors, enzymes, and peptide hormones.

“Host contaminant” or “Host cell contaminant” refers to biomolecules that are produced by the cells in which the product of interest is grown. The term may include various classes of host contaminants, such as host proteins and host DNA.

“Host protein” or “Host cell protein” or “HCP” refers to proteins that are produced by the cells in which the product of interest is grown. Such proteins represent one class of contaminants that must be removed from the product of interest.

“Antibody” refers to an immunoglobulin of the class IgG, IgM, IgA, IgD, or IgE derived from human or other mammalian cell lines, including natural or genetically modified forms such as humanized, human, single-chain, chimeric, synthetic, recombinant, hybrid, mutated, grafted, and in vitro generated antibodies. Antibodies may be produced by a single clone, in which case they are referred to as monoclonal, or from more than one clone, in which case they are referred to as polyclonal. IgG antibodies particularly refer to a class of antibodies referred to as immunoglobulin G, which may also exist as one or a mixture of subclasses, for example in humans as IgG₁, IgG₂, IgG₃, or IgG₄; or in mice as IgG₁, IgG_{2A}, IgG_{2B}, or IgG₃; or in rat as IgG₁, IgG_{2A}, IgG_{2B}, IgG_{2C}. Antibodies produced naturally or recombinantly in eukaryotic hosts may exist in a variety of glycosylated forms, while antibodies produced in non-eukaryotic hosts may exist in a variety of glycosylated and non-glycosylated forms. “Antibody” may also include composite forms including but not limited to fusion proteins containing an immunoglobulin moiety, or immunoconjugates created by synthetic linkage of an IgG to another functional moiety, including another antibody, an enzyme, a fluorophore or other signal generating moiety, biotin, a drug, or other functional moiety.

“Non-ionic organic polymer” refers to a naturally occurring or synthetic hydrocarbon composed of linked repeating organic subunits that lack charged groups. It may be linear, dominantly linear with some branching, or dominantly branched. Examples suitable to practice the embodiments disclosed herein include but are not limited to polyethylene glycol (PEG), polypropylene glycol, and polyvinylpyrrolidone (PVP). PEG has a structural formula HO—(CH₂—CH₂—O)ₙ—H. Examples include, but are not limited to compositions with an average polymer molecular weight ranging from less than 100 to more than 10,000 daltons.

“Tangential flow filtration (TFF)” refers to a method of membrane filtration in which fluid is forced through a space bounded by one or more porous membranes, where molecules small enough to pass through the pores are eliminated in the filtrate (sometimes the permeate), and molecules large enough to be rejected by the pores remain in the retentate. The name tangential flow particularly refers to the fact that the direction of fluid flow is roughly parallel to the membrane, as opposed to so-called dead-end filtration where flow is roughly perpendicular to the membrane.

“Tangential flow filtration membrane” refers to a porous membrane configured to practice the technique of tangential flow filtration (TFF). Such membranes are commonly configured in flat sheets, which may be stacked in to multiple membrane units; or extended sheets that are rolled to reduce the amount of space they occupy, referred to as spiral-wound membranes; or so-called hollow fibers where liquid flows through the lumen, liquids and sufficiently small solutes pass through the pores while larger solids are retained, and a plurality of which are typically housed in a cartridge format to facilitate handling. Standard terminology names microporous membranes and ultraporous membranes. The average pore size distribution of microporous membranes is generally published and typically includes porosities of 0.22 micron, and 0.45 microns, but can also include porosities of 1, 2, 5 microns and higher. The average pore size distribution of ultraporous membranes is generally not published but expressed in arbitrary terms based on an assumed equivalent size of a globular protein. A perfectly globular (spherical) protein might have a diameter of about 5 nm, but in practice, proteins of this molecular weight have been reported to have a hydrodynamic size of more than twice that (IgG, 11 nm hydrodynamic diameter). Because of the arbitrary nature of this standard, ultrafiltration membranes are sometimes described more precisely as nanofiltration membranes, or nanoporous membranes. Strictly speaking they include so-called microporous membranes with average porosities less than 1 micron, but are more commonly considered to include porosities of 100 nm or less, down to porosities of 10 nm or less. Both nanoporous and microporous membranes are commonly available with surfaces that are intended to be chemically inert, but also include membranes that have been chemically modified to create a surface capable of interacting with dissolved molecules by electrostatic or hydrophobic interactions, or other types or mixtures of chemical mechanisms, even including biological affinity. A TFF membrane suitable for retaining an IgG antibody in certain embodiments includes an electropositive membrane having a porosity that retains at least 50% of non-adsorbed solutes with a hydrodynamic diameter greater than a selected size but permits passage of non-adsorbed solutes with a hydrodynamic diameter less than the selected size where the selected size may be any amount between about 10 nm and about 15 nm.

“Tangential flow filtration module” or tangential flow filtration subunit refers to a functional component of the disclosed apparatuses and processes that employs a tangential flow filtration membrane with a pore size distribution that permits retention of at least 50% (or at least 60%, 70%, 80%, 90%, 95%, 99% or practically all) of the desired product, while permitting smaller contaminants to pass through the pores of the membrane. The disclosed apparatuses and methods differ fundamentally from traditional tangential flow filtration by integrating membrane fractionation with an adsorption function as defined below.

“Diavolume” refers to the ratio of the total volume of buffer added during an operation (such as tangential flow filtration) to the volume of the retentate.

“Adsorption subunit” or adsorption subunit refers to a functional component of the disclosed apparatuses and processes that employs a solid phase bearing a chemically interactive surface that can retain molecules of complementary character and through their elimination from a preparation, thereby achieve a degree of fractionation. The disclosed apparatuses and processes differ from traditional adsorption chromatography by integrating a size fractionation function by means of integrating membrane filtration function as defined above.

“Adsorptive chromatography” refers to one of the two basic types of chromatography commonly carried out for purification of biological products, the other being “non-adsorptive chromatography.” Non-adsorptive chromatography includes methods that sort entities by size, with specific examples including size exclusion chromatography and filtration through membranes with defined pore size distributions. In non-adsorptive chromatography, the ideal is for the contents of a given preparation to not interact chemically with the surfaces of the media used to mediate the fractionation. Physical formats for conducting adsorption chromatography include columns packed with porous particles, monoliths, and membranes in the form of flat sheets or hollow fibers, or combinations. Adsorptive chromatography includes methods that exploit surfaces that embody a particular type of chemistry that is intended to form stable associations with some species within a mixed preparation, and not with others; and/or, through manipulation of the chemical environment by means of altering buffer conditions, permit the fractionation of components according to the relative intensity with which they interact with the adsorbent. Many types of adsorption mechanisms are well known in the art. Anion chromatography involves the use of an electropositive adsorbent that binds molecules of strongly electronegative character but repels strongly electropositive molecules. Further fractionation can be achieved among bound species by increasing the concentration of salt and/or decreasing pH. The same principles are involved in cation exchange chromatography except that the charge on the solid phase is reversed (electronegative), so that adsorption favors species that are electropositive, while electronegative species tend to be repelled. Hydrophobic interaction chromatography uses hydrophobic adsorbents. Adsorbents of “multimodal” character comprise combinations of negative charges, positive charges, hydrophobic groups of various types, hydrogen bonds, and other mechanisms.

Traditional adsorption chromatography is practiced in two modes. “Bind-elute” mode is performed under conditions where the product of interest binds to the adsorbent. Unbound contaminants flow through the adsorbent and are thereby eliminated. The bound product is then dissociated from the adsorbent by changing the buffer conditions. This dissociation step is referred to as “elution.” Elution conditions are usually set so that contaminants that bind the adsorbent more strongly than the product remain bound after the product has been eluted. The other operational mode of adsorption chromatography is “flow-through” mode, which is performed under conditions where the product flows through the adsorbent, while contaminants that bind to the adsorbent are separated from the flow-through product.

All traditional-conventional adsorption chromatography materials—but not the disclosed apparatuses and processes—employ the same operational steps, regardless of their physical format or operating characteristics: the adsorbent is equilibrated to a defined set of conditions before contact with the preparation. The preparation is equilibrated to a defined set of conditions before contact with the adsorbent. The contact conditions define the “selectivity” which refers to which component(s) of a preparation bind to the adsorbent, and which do not. In bind-elute mode, the elution conditions define the selectivity of what species are eluted and which remain bound.

“Polynucleotide” refers to a biopolymer composed of multiple nucleotide monomers covalently bonded in a chain. DNA (deoxyribonucleic acid) and RNA (ribonucleic acid) are examples of polynucleotides. Polynucleotides can have a high propensity for formation of hydrogen bonds.

“Protein preparation” refers to any aqueous or mostly aqueous solution containing a protein of interest, such as a cell-containing cell culture harvest, a (substantially) cell-free cell culture supernatant, or a solution containing the protein of interest from a stage of purification.

“Virus” or “virion” refers to an ultramicroscopic (roughly 20 to 300 nm in diameter), metabolically inert, infectious agent that replicates only within the cells of living hosts, mainly bacteria, plants, and animals: composed of an RNA or DNA core, a protein coat, and, in more complex types, a surrounding envelope.

Embodiments herein provide various apparatus having instrument configurations suitable for purification of biological products from unpurified or partially purified preparations. Adsorption occurs in the disclosed apparatuses and processes but the operational approach is distinct because the behavior of product and contaminants in the system are constrained by the combination of an adsorption subunit with a tangential flow-based size fractionation subunit. The combination permits capabilities that are not possible with adsorption chromatography performed with traditional chromatography instruments or processes, not possible with tangential flow filtration instruments or processes, and not possible with other known combinations of adsorption chromatography and tangential flow filtration. The uniqueness of the system is further emphasized by the fact that the disclosed processing format cannot be performed with traditional-conventional chromatography apparatuses, and traditional chromatography processes cannot be performed on the disclosed apparatuses. The distinctions between traditional chromatographs-chromatography and the disclosed apparatuses and processes of the invention are illustrated throughout the description herein, including the Examples below. The Examples illustrate the unique format of certain embodiments of the invention by which the apparatus and processes may fractionate unpurified or partially purified preparations. Chemistry details in exemplary processes are provided to promote a clear understanding of the apparatus and methods, but in many embodiments of the invention such chemical details do not define the disclosed apparatus or methods performed with such apparatus.

In many embodiments of the invention, the disclosed apparatuses and processes are distinct from the apparatus and process formats employed in conventional chromatography, but can employ some of the same adsorption chromatography components. The physical forms of adsorption chromatography media used to practice certain embodiments of the disclosed methods/processes of the invention may include physical forms used to practice traditional adsorption chromatography methods, such as monoliths, membrane-adsorbers, and columns packed with porous particles, but they are applied in a distinct operational format. Adsorption chromatography media that may be employed in the adsorption subunits include the same kinds of media used for traditional adsorption chromatography processes, but they are applied in a distinct operational format. Adsorption chromatography media suitable for performing the disclosed methods/processes in certain embodiments include chromatography media for anion exchange chromatography, cation exchange chromatography, hydrophobic interaction chromatography, multimodal chromatography, affinity chromatography, or chromatography media for any other adsorptive mechanism. The buffers employed to control the selectivity of adsorption chromatography media are likewise similar to the buffers employed to control selectivity in traditional adsorption chromatography, but are applied in a distinct processing format. The disclosed methods/processes of certain embodiments disclosed herein likewise employ a uniform processing format distinct from traditional adsorption chromatography without regard to what products, protein or non-protein, are to be processed. Similarities between traditional chromatography and some components of the disclosed apparatuses and processes notwithstanding, the configuration of the disclosed apparatus, processing format, and results are distinct from apparatuses, processing formats, and results obtained with traditional adsorption chromatography. The apparatuses and processes of many embodiments of the invention are defined by the unique processing format and capabilities they permit, and the unique results obtained by applying them to purification of biological products and not by the type of adsorption chromatography media, physical form of adsorption chromatography media, buffers used to control selectivity of adsorption chromatography media, and not by the biological product or the preparation in which it resides.

The disclosed apparatuses and processes of certain embodiments are distinct from the apparatuses and process formats employed in conventional tangential flow filtration, but can employ some of the same tangential flow filtration components. The physical forms of tangential flow filtration media used to practice the disclosed methods/processes include physical forms used to practice traditional flow filtration, such as flat membranes, rolled (spiral wound), and hollow fibers, but they are applied in a distinct operational format. Tangential flow filtration media that may be employed in the tangential flow filtration modules include the same kinds of media used for traditional adsorption chromatography processes, such as cellulose-based or polyethersulfone (PES)-based, or employing other polymers, but they are applied in a distinct operational format. The buffers employed during tangential flow filtration may be likewise similar to the buffers employed to in traditional tangential flow filtration, but are applied in a distinct processing format. The disclosed methods/processes of certain embodiments likewise employ a uniform processing format without regard to what products, protein or non-protein, are to be processed. Similarities between traditional tangential flow filtration and some components of the disclosed apparatuses and processes notwithstanding, the configuration of the disclosed apparatus, processing format, and results are distinct from apparatuses, processing formats, and results obtained with traditional tangential flow filtration. The apparatuses and processes are defined by the unique processing format and capabilities they permit, and the unique results obtained by applying them to purification of biological products; not by the type of tangential flow filtration media, physical form of tangential flow filtration media, buffers, and not by the biological product or the preparation in which it resides.

In certain aspects of the invention, methods for purifying IgG monoclonal antibodies provide a useful example for how the disclosed embodiments operate as whole, and how conditions are altered to achieve target results. It will be apparent to those skilled in the art that similar approaches will be applicable to other IgG monoclonal antibodies, other antibodies, and non-antibody proteins, polynucleotides, and virus particles, for which the specific choice of inter alia tangential flow membrane porosity, number, surface chemistry, and physical format of adsorption subunits, and buffer conditions may be determined by the properties of the target product, but for which the execution of the methods will otherwise be the same or highly similar.

Experimental results indicate that with respect to the methods of certain embodiments of the invention provide beneficial results when an antibody preparation has first been clarified to reduce the content of chromatin by 90% or more. Such chromatin-reduction may be achieved by chromatin-directed clarification methods such as those described herein. One such method is performed by adding allantoin to a cell-containing or cell-free cell culture harvest in an amount equivalent to about 1% v/v. Allantoin addition is followed by addition of caprylic acid to a final concentration of about 0.4%, and the operating pH is reduced to about 5.3. The mixture is incubated stiffing for two hours at room temperature, then particles bearing the cationic chelating agent tris(2-aminoethyle)amine (TREN) are added in an amount of about 5% v/v. The mixture is incubated stiffing for four hours. Most of the solids are removed by centrifugation and the remainder by an optional depth filtration step. Alternatively, solids may be removed after treatment with fatty acid, without addition of TREN particles, but where the solids-free preparation is passed through a column packed with TREN particles, where the volume of TREN particles in the column is about 5% or the preparation volume to be processed. Alternatively, the column packed with TREN particles may be substituted with a column packed with UNOsphere Q particles, and the preparation processed by the known method of void exclusion anion exchange chromatography. Alternatively, caprylic acid may be replaced by other fatty acids, or electropositive hydrophobic agents such as ethacridine, methylene blue, or cetyltrimethyl ammonium bromide. Methods may also employ particles with different chemical surfaces, or alternative physical formats such as membranes, monoliths or others. Allantoin may be optionally omitted from the methods disclosed herein.

In some embodiments, the apparatus may be fitted with the following purification subunits: (i) a tangential flow filtration subunit equipped with a regenerated cellulose membrane with an average pore size sufficient to cause the membrane to retain a globular protein with a mass of about 30 kDa; (ii) a first adsorption subunit consisting of a monolith bearing a quaternary amino group such as used for performing the technique of anion exchange chromatography. A commercial example of such a monolith is a CIM QA monolith manufactured by BIA Separations. Conditions for adsorption of contaminants on the surface of the monolith are defined, as for all adsorption chromatograph methods, by routine experiments to identify the combination where the most contaminants bind but the IgG monoclonal antibody does not. As a general matter, the best conditions will be the highest pH at which the antibody does not bind in the absence of added salts. Conditions are understood to be different for every antibody because each has unique charge properties, but for many IgG1 monoclonal antibodies, an operating pH of 8.0 created by 50 mM Tris, pH 8.0 provides a good starting point. Once the conditions are identified for the adsorption subunit, the disclosed apparatus may be used to fractionate the preparation with the disclosed process. The preparation itself may be fairly crude in composition such as a clarified cell culture harvest, or it may be highly purified, such as the IgG fraction following purification in an earlier process step by protein A affinity chromatography, or any other degree of purity following some other previous fractionation step. The preparation is introduced into the apparatus under any chemical conditions that do not damage the antibody. The preparation may be in contact initially with only the TFF subunit (adsorption subunit off-line), during which the concentration of the IgG may be increased and the buffer conditions altered, and during which contaminants small enough to pass through the pores in the membrane are thereby eliminated. The adsorption subunit need not be equilibrated in advance, which is a fundamental distinction from traditional adsorption chromatography strictly requires that the adsorption chromatography medium be equilibrated before contacting the preparation. The next step is to put the adsorption subunit in line so that the IgG-containing retentate from the tangential flow filtration subunit passes through the adsorption subunit. In another fundamental distinction from traditional adsorption chromatography, the preparation need not be equilibrated before coming into contact with the adsorption subunit. Neither the preparation nor the adsorption subunit need be equilibrated before coming into contact with each other. In traditional adsorption chromatography, this would cause the method to fail. In the disclosed method, this is advantageous because it allows the chromatography adsorbent to be equilibrated in tandem with the preparation, which reduces buffer volume and process time. The buffer formulation is altered towards final conditions by means of diafiltration through the TFF subunit. As the preparation and the adsorption subunit approach final conditions, small contaminants continue to be eliminated through the pores of the membrane, while acidic contaminants begin to bind to the adsorption subunit. The process is complete when the fully equilibrated sample has passed at least once through the adsorption subunit. During the entire process, small contaminants have been eliminated through the pores in the membrane. This occurs to a lesser degree with traditional tangential flow filtration because the duration of the process is truncated so as not to inflate process time or volume. It is more efficient with the disclosed system because the extended time created by inclusion of the adsorption chromatography allows more small contaminants to be removed through the membrane. After processing, the preparation may be rinsed from the lines and harvested.

In some embodiments, the apparatus may be configured to support a more complex process. In one such example, the apparatus is fitted with the following purification subunits: (i) a tangential flow filtration subunit equipped with a regenerated cellulose membrane with an average pore size sufficient to cause the membrane to retain a globular protein with a mass of about 30 kDa; (ii) a first adsorption subunit with an aromatic hydrophobic group, such as a membrane-based device bearing phenyl groups on its surface, or a monolith-based device bearing butyl groups on its surface; (iii) a second adsorption subunit consisting of a monolith or a membrane bearing a quaternary amino group such as used for performing the technique of anion exchange chromatography; (iv) a third adsorption subunit bearing sulfo groups such as used for performing cation exchange chromatography. For this more complex system, conditions are for each of the adsorption subunits are established in the same fashion as for the previous simple example with only a single anion exchange adsorption subunit. In each case, the conditions are identified that permit binding by the largest diversity of contaminants, without binding a significant amount of antibody. Conditions for the hydrophobic interaction adsorption subunit commonly include elevated concentrations of kosmotropic salts, or very low concentrations of salts. In either case, conditions are set so that contaminants, especially including aggregates, bind to the adsorption subunit, but binding of IgG is minimal. For one specific monoclonal antibody (see Examples 1-8), two appropriate sets of conditions were identified: 0.4 M sodium citrate, 50 mM Tris, pH 8.0, and 12.5 mM sodium citrate, 50 mM MES, pH 6.0. Conditions may also be established for the cation exchange chromatography module. In this case, as with anion exchange and hydrophobic interaction, conditions were set to bind contaminants but not antibody. The particular conditions for the SO₃ monolithic adsorption subunit were 5 about mM NaCl, about 50 mM Tris, at about pH 8.0. Conditions for the anion exchange step are as described in the previous example. It will be recognized that the adsorptive chemistries and conditions are largely the same as used with conventional adsorption chromatography used in so-called flow-through mode, but that the disclosed apparatus and methods achieve better results because of the continuous elimination of small contaminants through the pores of the tangential flow filtration subunit for the entire duration of the process. This example employing multiple adsorption steps highlights another unique feature of certain embodiments of the invention: combinations of different adsorption chromatography chemistries within a single integrated apparatus, within the confines of a single unit operation. The term single unit operation refers to a case where an unpurified preparation is introduced into the system and the process is considered complete when the sample is removed from the system. The disclosed apparatus and methods is unique among combinations of tangential flow filtration and adsorption chromatography in its capability to sequentially process a preparation by multiple distinct adsorption methods, all during which contaminants are being removed by passage through the tangential flow filtration membranes. A particular benefit of running such processes in a single unit operation is that they support higher product recovery. This is because in systems that support only one adsorption method to be run at a time, the product must be removed after each step before it can be introduced to the next. Some product is invariably lost at each transfer, so eliminating such transfers contributes to higher product recovery.

In some embodiments where the apparatus is fitted with multiple adsorption subunits for the purpose of performing multiple adsorption methods within a single unit operation, the sequence of adsorption methods may be varied, and some sequences may prove advantageous over others. In certain embodiments, the apparatus provided will be configured to permit different adsorption methods to be individually combined with the tangential flow filtration subunit separately and in any order desired by the user. As a matter of principle, a sequence in which contaminants that may interfere with a given adsorption step are removed before that adsorption step will be advantageous. By the same logic, a sequence in which contaminants that might stress the capacity limitations of a given adsorption step are removed before that adsorption step will be advantageous. In practice the sequence of such steps will depend on the contaminant content of the sample introduced into the system, and the preferred sequence, if any, can be identified by simple experimentation.

An example, employing only two adsorption subunits is illustrated here: A chromatin deficient IgG preparation is concentrated on the tangential flow filtration module to a concentration of about 20 g/L. Most contaminants smaller than about 100 kDa will be eliminated at this step by their passage through the membrane in the tangential flow filtration subunit. The concentrated IgG may optionally be treated to inactivate potentially remaining virus, for example by buffer exchanging the IgG preparation into 100 mM acetic acid, 1.7 M NaCl, pH 3.5. Most of the remaining contaminants smaller than about 100 kDa will be eliminated at this step. The first adsorption subunit (an axial flow phenyl membrane adsorber) is placed in line and the IgG preparation is buffer exchanged into 25 mM sodium phosphate, 1.7 M NaCl, pH 7.2. The adsorption subunit and the tubing leading into and out of it are rinsed with 25 mM sodium phosphate, 1.7 M NaCl, pH 7.2. The first adsorption subunit is put off-line and the second adsorption subunit (an axial flow QA monolith) is put in line. The IgG preparation is buffer exchanged through the tangential flow filtration module to 25 mM Tris, pH 8.0. The second adsorption subunit is rinsed with 25 mM Tris, pH 8.0, and the unit is put off line. The IgG preparation is buffer exchanged into the final formulation or pre-formulation buffer of choice. The main body of IgG is collected and the apparatus is rinsed with the final formulation or pre-formulation buffer of choice to enable collection of antibody remaining within the apparatus.

The above conditions were developed for a Herceptin biosimilar antibody, and permit the multistep purification of the clarified harvest to be conducted as a single unit operation. There is no requirement to remove antibody from the unit for intermediate processing (buffer exchange) or to conduct the second adsorption step. Multiple processing steps can be conducted within the apparatus without removing the preparation from the apparatus.

Purification of IgG also represents a platform from which to appreciate variations of the technique that may be applied. In one such embodiment, chromatin-directed clarification is performed by adding allantoin to a cell-containing or cell-free cell culture harvest in an amount equivalent to about 1% v/v. Allantoin addition is followed by addition of methylene blue to a concentration of 0.025%, and the operating pH is raised to 8.0. The mixture is incubated stiffing for 2 hours at room temperature, then particles bearing the cationic chelating agent tris(2-aminoethyle)amine (TREN) are added in an amount of 5% v/v. The mixture is incubated stiffing for 4 hours. Most of the solids are removed by centrifugation and the remainder by an optional depth filtration step, such as with a PC1 depth filter (Sartorius). In actual usage, a virus filtration step may also be applied at this point. In another such embodiment, the membrane in the at least one tangential flow filtration subunit is a polyethersulfone (PES) membrane with an average pore size corresponding to a hypothetical globular protein with a mass of about 50 kDa. In another such embodiment, the membrane in the at least one tangential flow filtration subunit is a regenerated cellulose membrane with an average pore size corresponding to a hypothetical globular protein with a mass of about 30 kDa. In some embodiments, adequate purification may be achieved with only a single adsorption step, performed with the integrated at least one adsorption subunit. In some such embodiments, the preparation-contact surface of that subunit may bear positive charges, endowing it with the ability to perform as an ion exchanger. In some embodiments, the positive charges may be endowed by a primary amino group, a secondary amino group, a tertiary amino group, a quaternary amino group, or combinations of the foregoing. In some such embodiments the surface of an electropositive adsorption subunit may further embody the ability to participate in other types of chemical interactions, such as hydrophobic interactions, hydrogen bonding, and/or metal affinity. In some such embodiments, the surface of the adsorption subunit is functionalized with tris(2-aminoethyl)amine (TREN). In some embodiments, an additional adsorption subunit may be desired, for example for the purpose of reducing the content of aggregates in the preparation. Surfaces of such subunits may particularly include hydrophobic residues in addition to residues that participate in hydrogen bonds. In embodiment, a harvest conditioned to remove chromatin as described is contacted with the apparatus and the IgG concentrated to about 60% g/L by the at least one tangential flow filtration subunit equipped with a regenerated cellulose membrane with an average pore size corresponding to a hypothetical globular protein with a mass of 30 kDa. As soon as all of the harvest has been introduced, the input buffer is changed to 50 mM acetic acid, 1.7 M NaCl, pH 3.5. When the preparation reaches these conditions, a first adsorption subunit consisting of a membrane device bearing hydrophobic phenyl groups is place in line with the at least one tangential flow filtration subunit. Up until this point, contaminants smaller than the pore size distribution of the membrane in the at least one tangential flow filtration have been continuously removed by passage through the pores of the membrane. It will be recognized that the high salt formation likely also mediates a dissociative effect on non-specific interactions that may exist under physiological conditions between contaminants and the desired IgG, and/or between contaminants and the internal contact surfaces of the apparatus as a whole. A buffer is introduced containing 50 mM Hepes, 1.7 M NaCl, pH 7.0. When the preparation as a whole embodies these conditions, aggregates will have been removed by binding to the surface of the phenyl membrane adsorption subunit. The unit is rinsed with 50 mM Hepes, 1.7 M NaCl, pH 7.0 and put off-line. At the same time the first adsorption subunit is put off-line, a second adsorption subunit bearing electropositive charges is placed in-line. In one such embodiment, the second adsorption subunit is a monolith bearing TREN. In one such embodiment, the second adsorption subunit is a monolith bearing quaternary amino groups. In one such embodiment, the second adsorption subunit is a monolith bearing ethylene diamine groups. In one such embodiment, the second adsorption subunit is a microporous membrane-based device bearing quaternary amino groups. In one such embodiment, the second adsorption subunit is a microporous membrane-based device bearing polyallylamine groups. In one such embodiment, the second adsorption subunit is a hydrogelatinous-filled device bearing quaternary amino groups. In one such embodiment, the second adsorption subunit is a microporous hollow-fiber membrane-based device bearing a network of electropositive groups. In some embodiments, a buffer consisting of 50 mM Tris, pH 8.0 is introduced and the preparation gradually transitions to that composition, during which small contaminants continue to be eliminated through the membrane of the at least on tangential flow filtration subunit. As of the point that the preparation as a whole achieves the conditions of 50 mM Tris, pH 8.0, acidic contaminants are understood to have bound to the surface of the second adsorption subunit, achieving there elimination. At this point the second adsorption subunit is put off line. The purified IgG may optionally be collected or buffer exchanged into a pre-formulation buffer and then collected. It will be apparent that the apparatus may be fitted with a wide variety of tangential flow subunits and/or chromatography subunits without departing from the essential features and functions of the disclosed apparatus and methods.

In some embodiments, the first fractionation step following concentration of the desired IgG may be a precipitation step. In some embodiments, a precipitation step may follow a dissociation step. The latter may be preferred in some embodiments because precipitation steps bear the inherent risk of stabilizing otherwise transient non-specific associations between contaminants and the desired product, or between contaminants and an internal contact-surface of the apparatus. In one embodiment selected particularly to illustrate the principal of such an application, the conditioned preparation is first concentrated through the at least one tangential flow filtration subunit, then a dissociating buffer is introduced such as 1 M guanidine-HCl, pH 5.5. During the concentration and dissociation stages, most of the contaminants will be eliminated through the membrane of the at least one tangential flow filtration subunit. A precipitation buffer is introduced, in one embodiment consisting of 1.3 M citric acid pH 6.0. As the proportion of precipitating buffer increases in the preparation as a whole, the IgG precipitates. The flow path is then switched to direct the preparation through a second tangential flow filtration subunit with a pore size appropriate for retaining precipitates, but not soluble proteins. After the precipitate has been washed sufficiently with 1.3 M citric acid, pH 6.0 that a sufficient quantity of soluble contaminants are judged to have been eliminated through the membrane, the flow path is switched back so that flow is directed through the tangential flow filtration subunit with the membrane suitable to retain the IgG in soluble form, while still allowing the passage and elimination of smaller contaminants. The input buffer is changed to 50 mM Tris pH 8.2 is introduced. At an intermediate point where the precipitates have become fully resolubilized, the at least one adsorption subunit, in some embodiments consisting of a monolith bearing quaternary amino groups, is placed in line. Small contaminants that may be resolubilized along with the desired antibody are removed by passage through the membrane of the at least one tangential flow filtration device. At the time when the preparation as a whole resides in 50 mM Tris, pH 8.2, remaining acidic contaminants are understood to have bound to the at least one adsorption subunit and thus have become eliminated. The absorption chromatography subunit is rinsed with clean 50 mM Tris, pH 8.2 to recover the desired IgG, and the adsorption subunit is put off line. The purified IgG may optionally be collected, or buffer exchanged into a preformulation buffer, then collected. In either case, trace levels of small contaminants continue to be removed by passage through the at least one tangential flow filtration device for the entire duration of the process. It will be apparent that precipitation-based fractionation of other desired products, including other proteins, viruses, and virus-like species can be performed in essentially the same manner, though optionally with different membranes and adsorption subunits.

Purification of IgG also serves as a platform to illustrate how the disclosed apparatuses and methods may be integrated with traditional chromatography apparatuses and methods. In some embodiments, purification need not be performed exclusively with the disclosed apparatus and methods. Some purification steps can be performed with the disclosed apparatus and methods, while other steps can be performed with traditional chromatography apparatuses and methods, or other novel chromatography apparatuses and/or methods. In one such embodiment, initial purification of an IgG monoclonal antibody is performed with protein A affinity chromatography conducted on a traditional chromatograph using traditional protein A affinity chromatography media packed in a column. Protein A chromatography may then be followed by anion exchange chromatography performed on the disclosed apparatus, such as in FIG. 1, using the disclosed methods, which produce several valuable benefits over traditional chromatography despite being restricted to only an anion exchange chromatography step. For example, where the traditional post-protein A sample must be equilibrated to anion exchange conditions before contacting one with the other, and the anion exchange column must be also be equilibrated, with the disclosed apparatus and methods, neither need to be equilibrated prior to contact. Further, equilibration of traditional post-protein A material is frequently done by dilution, which increases sample volume, and because of that, the dilution frequently does not go to the lowest possible salt concentration. With the disclosed apparatus, salt concentration can be reduced as low as desired without dilution. Further, where the large amount of contaminating host proteins in post-protein A material burden the capacity of a follow-on traditional anion exchange step, with the present apparatus and methods, most of those contaminants can be eliminated through the pores of the tangential flow filtration module before the anion exchange adsorption subunit is placed in line, thereby conserving the capacity of the anion exchanger and potentially permitting the anion exchanger to process a larger volume of IgG, at a lower material cost per unit of IgG. Further, the traditional anion exchange in the mode applied with IgG purification removes contaminants that bind to the anion exchanger, but does not remove small contaminants that fail to bind to the anion exchanger. The disclosed apparatus and methods remove both. Acidic contaminants still bind to the anion exchanger, while small unbound contaminants are eliminated through the pores in the tangential flow filtration module.

Purification of IgM represents a platform from which to appreciate how the method may be adapted to a non-IgG protein. In one such embodiment, the conductivity of a cell culture harvest is increased to about 20 mS/cm by addition of NaCl. This is done to prevent inadvertent binding and IgM loss due to interactions with soluble or insoluble electropositive or electronegative organic ions to be subsequently added as a conditioning step prior to contacting the preparation with the apparatus. pH is adjusted to 6.0 for the same reason. With other non-IgG proteins conductivity and pH may be adjusted to different values, higher or lower, according to the properties of the desired protein. In some embodiments, allantoin is added to the harvest in an amount of 1% v:v, followed by the addition of ethacridine or methylene blue at 0.025%, or a combination of the two where the combined concentration is 0.025%, or cetyltrimethylammonium bromide at a concentration of 0.025%, or a combination of cetyltrimethylammonium bromide with ethacridine and/or methylene blue where their combined concentration is 0.025%, and allowed to incubate stiffing for 60%-120 minutes. In some embodiments, functionalized particles such as particles functionalized with TREN are added, for example in an amount of 2-5% v:v and the mixture incubated for 2-4 hours or more, then the solids are removed by any expedient method. The conditioned preparation is contacted with the disclosed apparatus, such as illustrated in FIG. 1, where the first step is to reduce the volume of the preparation so that the approximate concentration of the desired product is 20 g/L, or 30, or 40, or 50 g/L or a higher concentration if permitted by the inherent solubility and viscosity of the desired protein. Due to the large size of IgM, at a about 1 million Daltons and with a hydrodynamic diameter of 22-25 nm, the membrane housed in the tangential flow filtration subunit may be have larger pores than required for purification of a smaller desired protein such as IgG. For example, it may be possible to employ a membrane with an average pore size corresponding with a hypothetical globular protein with a mass of 100 kDa, or 300 kDa. However, experimental data indicate that competitive results can be obtained with membranes with much smaller pore size distributions, such as membranes with an average size corresponding a hypothetical globular protein with a mass of 50 kDA, or 30 kDa, or even 10 kDa. In many cases, experimental data show that membranes constructed of regenerated cellulose support a higher degree of purification than membranes constructed of polymers such as polyethersulfone. After concentration, the preparation may then optionally be treated to achieve virus inactivation or with a step containing agents to dissociate contaminant species that may be nonspecifically associated with the desired protein. Without requirement to pre-equilibrate the preparation for conditions to bind contaminants to the at least one adsorption subunit, the preparation is contacted with the at least one adsorption subunit. In some embodiments, the at least one adsorption subunit may bear positive charges on its surface. Then, still in contact with the at least one adsorption subunit, the preparation is buffer exchanged through the contiguous at least one tangential flow filtration subunit, to the conditions that bind the most contaminants without binding an excessive amount of the desired protein. The desired protein is then cleared from the system and collected. The protein may alternatively be purified in a version of the apparatus including two adsorption subunits. In one such embodiment, one adsorption subunit may bear negative charges while the other bears positive charges. It will be apparent that this same general approach may be applied to any recombinant proteins, where the conditions for preparing a cell culture harvest are customized to the properties of the desired protein, the porosity of the membrane is chosen according to the size of the desired protein, and the chemical surface of the adsorption subunit or subunits is/are chosen according to the properties of the desired protein and contaminant species. It is within the purview of a person of ordinary skill in the art to select membranes of appropriate composition and porosity, adsorption subunits, and chemical conditions to perform the various steps of the overall purification process.

Purification of very-large DNA plasmids, such as used for gene therapy, provides another platform to describe application of the apparatus. In some embodiments, conditioning of the sample involves clarification through centrifugation and/or filtration, but will generally avoid agents used to remove chromatin since DNA is a component of chromatin. Conditioning may optionally include treatment with agents that embody electronegative and/or hydrophobic properties. In some embodiments, the first step upon contacting the preparation with the apparatus will be to concentrate the DNA, during which contaminants smaller than the pore size distribution of the membrane will be eliminated through the membrane of the at least one tangential flow filtration subunit. The membrane can be selected to embody a pore size distribution sufficient to retain the DNA plasmid. The larger the pore size distribution the larger the spectrum of contaminants that can be pass through, but membranes with a smaller pore size distribution than necessary to retain the DNA plasmid may also support adequate performance. As soon as all of the harvest has been introduced, a dissociating buffer is introduced and the preparation continues to be cycled through the system. In some such embodiments, the dissociating buffer may include a high concentration of a neutral or chaotropic salt, and/or a surfactant, and/or urea, and/or other dissociating agents, intended to dissociate the DNA plasmid from contaminants with which it may be non-specifically associated, and/or to dissociate contaminants which may be associated with surfaces inside the apparatus, and thereby facilitate their removal by passage through the membrane of the at least one tangential flow filtration subunit. With the preparation still resident in dissociating buffer, the at least one adsorption subunit may be placed in line. In one series of embodiments where the internal preparation-contact surface of the adsorption subunit is functionalized to embody elevated hydrophobicity, the buffer is then exchanged to a formulation that promotes hydrophobic interactions of protein contaminants, if any are present, such as a formulation containing a precipitating salt, like sodium or ammonium sulfate, sodium or potassium citrate, or potassium phosphate, in some embodiments at a neutral to mildly alkaline pH. As of the point where the preparation as a whole becomes equilibrated to these conditions, contaminants that bind to the adsorption subunit are thereby eliminated. The adsorption subunit is rinsed with clean buffer of the same formulation to retrieve DNA from the internal volume of the unit, the at least one adsorption subunit is put off-line, and a second adsorption subunit is optionally put in line. In one series of embodiments where the internal preparation-contact surface of the adsorption subunit is functionalized to embody a negative charge, the buffer is then exchanged to a low pH, low conductivity buffer such as 0.05 M acetic acid, pH 4.0. As of the point where the preparation as a whole becomes equilibrated to these conditions, contaminants less acidic than the DNA may bind to the second adsorption subunit and be thereby removed. The second adsorption subunit is then rinsed with clean 0.05 M acetic acid, pH 4.5, then the subunit is put-off line. The purified DNA plasmid may then be collected from the system, or optionally exchanged into a pre-formulation or final formulation buffer prior to being collected.

Purification of virus or virus-like particles provides another platform to appreciate the functional features of the apparatus. As with DNA, and sometimes to an even greater extent, conditioning a harvest to remove chromatin involves a high risk of removing the desired virus as well. Lipid-enveloped viruses in particular are very likely to be inadvertently removed by conditioning methods based on chromatin removal. Conditioning the sample may therefore often be limited to physical methods such as centrifugation and/or filtration. In some embodiments, the first step will be to concentrate the virus preparation to a smaller volume by means of the at least one tangential flow filtration unit, equipped with a membrane with a pore size distribution adequate to retain the virus. In many embodiments, the next step will be to introduce a dissociating buffer as described previous for DNA purification. As a general matter, the most strongly dissociating formulation that does not permanently render the virus unsuitable for its intended use will be preferred. With the virus still resident in dissociating buffer, the at least one adsorption subunit may be put in line, and a buffer introduced that will convert the conditions within the preparation to the conditions of the buffer, where those conditions are also designed to cause contaminants not already eliminated through the pores of the membrane in the at least one tangential flow filtration subunit, to bind to the surface of the at least one adsorption subunit. As a group, viruses and virus-like particles embody a much broader diversity of surface chemical features than proteins and DNA combined, but common chromatography surfaces such negative, charges, positive charges, hydrophobic residues, and combinations thereof may be employed. As a general matter, contamination of virus preparations by DNA released from cells killed by the virus during culture is a serious problem, especially to the extent that the DNA may be bound to the desired virus species. An adsorption subunit bearing positive charges may therefore be preferred to serve as an at least one adsorption subunit.

It will be apparent that many configurations apart from the Figures may be conceived that conserve the essential features of the disclosed apparatus. In one or more such embodiments, components of the disclosed apparatus may be uncoupled and certain functions performed manually on a step-by-step basis.

### EXAMPLES

Example 1. Chromatin was extracted from an IgG-containing cell culture harvest containing 259,218 ppm host protein contaminants and 21% aggregates. Chromatin was extracted by addition of 1% allantoin, 0.4% caprylic acid, pH 5.2; incubated for 2 hours, following which electropositive metal affinity particles (TREN 40 high, Bio-Works) were added to a volumetric proportion of 4%, and incubated mixing for an additional 4 hours, then solids were removed by passing the preparation through a depth filter (Sartorius PC1). Host proteins were reduced to 308 ppm and aggregates to less than 0.05%. A prototype apparatus was configured with a single tangential flow filtration subunit and a single adsorptive subunit. The tangential flow filtration subunit was equipped with a polyethersulfone (PES) membrane with an average pore size corresponding to a hypothetical globular protein with a hydrodynamic diameter of 50 kDa (Millipore). The adsorption subunit was a strong anion exchange monolith (CIM QA, BIA Separations). The conditioned harvest was concentrated to about 20 mg/mL through the tangential flow filtration subunit with the adsorption subunit off-line. The adsorption subunit was put in line with the tangential flow filtration subunit, and the buffer was exchanged to 50 mM Tris, pH 8.0. When the buffer flowing through the system reached the same pH and conductivity as the input buffer, the adsorption subunit line was rinsed with clean 50 mM Tris, pH 8.0, and the IgG collected. Regulatory standards for human-injectable therapeutic antibodies require host proteins to be reduced to 100 ppm or less, and aggregates to 1% or less. Three-step purification processes consisting of protein A affinity chromatography, cation exchange chromatography, and anion exchange chromatography are generally able to meet these requirements but seldom exceed them by a substantial margin. The disclosed methods reduced aggregates to less than 0.01% and host proteins to 11 ppm. An experimental control was run in which cell culture harvest was prepared using only centrifugation and microfiltration. It reduced aggregates to 2.14% and host proteins to 78,698 ppm, highlighting the contribution of chromatin extraction in advance of processing the preparation with the disclosed apparatus.

Example 2. The chromatin-extracted harvest of example 1 was applied to a prototype apparatus with the same tangential flow filtration subunit as in Example 1, but the adsorption subunit was replaced with a Sartobind Phenyl membrane adsorber (Sartorius). The conditioned harvest was concentrated to about 20 mg/mL with the adsorption subunit in line. The phenyl adsorption subunit was put in line and the buffer exchanged to 50 mM HEPES, 1.7 M NaCl, pH 7.0. When the buffer flowing through the system reached the same pH and conductivity as the input buffer, the phenyl adsorption subunit line was rinsed with clean 50 mM HEPES, 1.7 M NaCl, pH 7.0, and the adsorption unit was set off-line. The IgG collected and analyzed. Aggregates were reduced to less than 0.01% and host proteins were reduced to 9 ppm.

Example 3. The chromatin-extracted harvest of Example 1 was applied to a prototype apparatus with the same tangential flow filtration subunit and the same adsorption subunit (QA monolith), but with an additional adsorption subunit in the form of a Sartobind Phenyl membrane adsorber (Sartorius). The conditioned harvest was concentrated to about 20 mg/mL with no adsorption units in line. The phenyl adsorption subunit was put in line and the buffer exchanged to 50 mM HEPES, 1.7 M NaCl, pH 7.0. When the buffer flowing through the system reached the same pH and conductivity as the input buffer, the phenyl adsorption subunit line was rinsed, and switched off-line at the same time the QA monolith adsorption subunit was put on line. The buffer was exchanged to 50 mM Tris, pH 8.0. When the buffer flowing through the system reached the same pH and conductivity as the input buffer, the QA monolith adsorption subunit line was rinsed, and the IgG collected. Aggregates were reduced to less than 0.01% and host proteins were reduced to 1 ppm.

Example 4. The experiment of Example 3 was conducted with the same apparatus, materials and conditions, except substituting different anion exchange adsorption units in place of the QA monolith. Substitution of a Sartobind Q membrane adsorber (Sartorius) resulted in reduction of host protein to 1 ppm. Substitution of a salt tolerant interaction chromatography membrane adsorber (Sartorius) resulted in reduction of host protein to 1 ppm. Substitution of a DEAE monolith (BIA Separations) resulted in reduction of host protein to 1 ppm. Substitution of an EDA monolith (BIA Separations) resulted in reduction of host protein to 1 ppm. Aggregate levels were reduced to less than 0.01% in all experiments.

Example 5. The experimental format of Example 2 was reproduced except setting up the tangential flow filtration subunit with a cellulose membrane with a membrane with an average pore size corresponding to a hypothetical globular protein with a hydrodynamic diameter of 30 kDa (Millipore). Host protein contamination in the purified IgG was reduced to 2 ppm. Aggregates were less than 0.01%.

Example 6. The experimental format of Example 2 was reproduced except including a second adsorption subunit in the form of an EDA monolith. Host protein was undetectable. Aggregates were less than 0.01%.

Example 7. An IgG-containing cell culture harvest containing 277,433 ppm host protein contaminants and 23% aggregates was partially purified by protein A affinity chromatography, which reduced host protein content to 1,074 ppm, and aggregates to 1.1%. This step also served the purpose of removing most of the chromatin. The preparation was applied to a prototype apparatus configure with a single tangential flow filtration subunit equipped with a cellulose membrane with an average pore size corresponding to a hypothetical globular protein with a hydrodynamic diameter of 30 kDa (Millipore); and two adsorption subunits consisting or a phenyl membrane adsorber and a QA monolith. The partially purified IgG from the protein A step was applied to the apparatus and further purified as described in Example 2. Host protein was undetectable in the collected IgG, and aggregates were less than 0.01%.

Example 8. An IgG-containing cell culture harvest containing 243,997 ppm host protein contaminants and 24% aggregates was treated to extract chromatin by addition of 1% allantoin, 0.4% caprylic acid, pH 5.2; incubated for 2 hours, following which electropositive metal affinity particles (TREN 40 high, Bio-Works) were added in an amount of 4%, and incubated mixing for an additional 4 hours, then solids were removed by passing the preparation through a depth filter (Sartorius PC1). Host proteins were reduced to 305 ppm and aggregates to less than 0.05%. The antibody was further purified by cation exchange chromatography, reducing the host protein to 5 ppm, which was applied to a prototype apparatus including a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). Host protein was undetectable. Aggregates were less than 0.01%.

Example 9. An IgG-containing cell culture harvest containing 243,997 ppm host protein contaminants and 24% aggregates was treated to extract chromatin by addition of 1% allantoin, 0.4% caprylic acid, pH 5.6; incubated for 2 hours, following which electropositive metal affinity particles (TREN 40 high, Bio-Works) were added in an amount of 4%, and incubated mixing for an additional 4 hours, then solids were removed by passing the preparation through a depth filter (Sartorius PC1). Host proteins were reduced to 3,551 ppm and aggregates to less than 0.5%. The antibody was further purified by precipitation with 1.8 M ammonium sulfate, reducing the host protein to 1,423 ppm, which was applied to a prototype apparatus with a single tangential flow filtration subunit equipped with a PES membrane with an average pore size corresponding to a hypothetical globular protein with a hydrodynamic diameter of 50 kDa (Millipore), and a single adsorption subunit with a strong anion exchange monolith (CIM QA, BIA Separations). Host protein contamination in the collected IgG was reduced to 12 ppm. Aggregates were less than 0.05%.

Example 10. An IgG-containing cell culture harvest with 369,422 ppm host protein contamination, 11% aggregates, and about 10% free light chain was applied processed by protein A to reduce the chromatin load and partially purify the antibody. Host protein contamination was reduced to 1,873 ppm, aggregates to 1.12%, and free light chain to 0.4%. The preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). The preparation was initially processed by passing it through the apparatus with the adsorption subunit off line, during which time the buffer was 50 mM Hepes, 150 mM NaCl, pH 7.0. This reduced host protein contamination to 350 ppm, and reduced free light chain to 0.2%, but aggregate content increased to 1.70%. The adsorption subunit was set in line and the buffer changed to 20 mM Tris, pH 8.0. Host protein contamination was reduced to 8 ppm, aggregates to 0.55%, and free light chain to 0.19%.

Example 11. Chromatin was extracted from an IgG-containing cell culture harvest with 293,158 ppm host protein contamination, 23.35% aggregates, and about 12% free light chain by treating it 1% allantoin, 0.4% caprylic acid, pH 5.4; incubating it for 2 hours, removing the solids by microfiltration, then passing it through a column packed with Bio-Works TREN (high) with a volume of 5% the volume of the original cell culture harvest. Host protein contamination was reduced to 3,565 ppm, aggregates to 2.1%, and free light chain to 1.72%. The preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). Host protein contamination in the collected IgG amounted 563 ppm, aggregate content increased to 2.7%, and free light chain appeared to increase to 2.37%. The preparation was subdivided into 3 portions for separate subsequent processing. The portion treated by hydroxyapatite chromatography (CHT Type I, 40 micron, Bio-Rad) in bind-elute mode reduced host proteins to 113 ppm, aggregates to 1.22%, and free light chain to 1.7%. The portion treated by chromatography on a column packed with an electronegative multimodal chromatography medium (HCX, Merck-Millipore) in bind-elute mode reduced host proteins to 18 ppm, aggregates to 1.61%, and free light chain to 0.49%. The portion treated by chromatography on an electropositive multimodal chromatography medium (Capto adhere, GE Healthcare) in bind-elute mode reduced host protein contamination to 3 ppm, aggregates to 0.47%, and free light chain to the point of being undetectable.

Example 12. The chromatin-extracted preparation from example 11 was applied to a prototype apparatus configured in the same manner as in example 11, except that the QA anion exchange monolith was replaced by an SO3 cation exchange monolith and the buffer was changed to 50 mM Tris, 25 mM NaCl, pH 8.0 so that the antibody would not bind significantly to the cation exchanger. Host protein contamination was reduced from 3,565 ppm to 2,852 ppm, aggregates were reduced from 2.01% to 0.96%, and free light chain content appeared to increase from 1.72% to 2.75%. The preparation was subdivided into 3 portions for separate subsequent processing. The portion treated by hydroxyapatite chromatography (CHT Type I, 40 micron, Bio-Rad) in bind-elute mode reduced host proteins to 1,354ppm, aggregates to 0.15%, and free light chain to 2.80%. The portion treated by chromatography on a column packed with an electronegative multimodal chromatography medium (HCX, Merck-Millipore) in bind-elute mode reduced host proteins to 147 ppm, aggregates to 0.96%, and free light chain to 0.43%. The portion treated by chromatography on an electropositive multimodal chromatography medium (Capto adhere, GE Healthcare) in bind-elute mode reduced host protein contamination to 72 ppm, aggregates to 0.16%, and free light chain to the point of being undetectable.

Example 13. The chromatin-extracted preparation from example 11 was applied to a prototype apparatus configured in the same manner as in example 11, except that the QA anion exchange monolith was replaced by a hydrophobic butyl monolith. The preparation was divided into 3 portions. One proportion was buffer exchanged on the apparatus to 20 mM MES, pH 6.0, under which the antibody did not bind to the adsorbent. Host protein contamination of the collected antibody was reduced from 3,565 ppm to 1,894 ppm, aggregate content appeared to increase slightly from 2.01% to 2.17%, and free light chain content appeared to increase from 1.72% to 2.51%. One portion was buffer exchanged on the apparatus to 20 mM Hepes, pH 7.0. Host protein contamination of the collected antibody was reduced from 3,565 ppm to 2,525 ppm, aggregate content was reduced from 2.01% to 1.61%, and free light chain content appeared to increase from 1.72% to 2.43%. One portion was buffer exchanged on the apparatus to 20 mM Tris, pH 8.0. Host protein contamination of the collected antibody was reduced from 3,565 ppm to 3,095 ppm, aggregate content was reduced from 2.01% to 0.82%, and free light chain content appeared to increase from 1.72% to 3.17%.

Example 14. An IgG-containing mammalian cell culture supernatant with host protein contamination at 393,093 ppm and 10.96 aggregates was applied to a column of protein A to enrich the IgG and reduce chromatin content. Host protein content was reduced to 2,058 ppm and aggregates were reduced to 1.22%. NaCl was dissolved to a final concentration of 1M and the preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and two adsorption subunit plumbed in series equipped with a strong anion exchange monolith and a hydroxyl-bearing monolith, respectively (CIM QA, CIM OH, BIA Separations. The preparation was buffer exchanged with the adsorbent chromatography subunits off-line to 20 mM Tris, pH 8.0, during which host protein contamination was further reduced to 1242 ppm while aggregates increased to 1.33%. The adsorbent chromatography subunits were placed in-line and the buffer and the sample recirculated through the system. Host contaminants were reduced to 8 ppm and aggregates to 0.42%.

Example 15. Chromatin was extracted from an IgG-containing cell culture harvest with 260,184 ppm host protein contamination and 26.05% aggregates by treating it with 1% allantoin, 0.4% caprylic acid, pH 5.4; incubating it for 2 hours, removing the solids by microfiltration, then passing it through a column packed with Bio-Works TREN (high) with a volume of 5% the volume of the original cell culture harvest. Host protein contamination was reduced to 5,378 ppm and aggregates to 3.98%. IgG concentration was reduced from the original 0.71 mg/mL to 0.59 mg/mL The preparation was applied to a column packed with electronegative multimodal particles (HCX, Merck-Millipore), the column washed, and the IgG was eluted by salt gradient, reducing host protein content to 490 ppm and aggregates to 2.06%. IgG concentration was 5.69 mg/mL The HCX-eluted preparation at about 0.7 M NaCl was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). The adsorption subunit was initially off-line. The preparation was concentrated to an IgG concentration of 34 mg/mL, during which host protein concentration was reduced to 297 ppm and aggregates were at 2.17%. The adsorption subunit was put in line and the preparation recirculated during infusion of 50 mM Tris, pH 8.0 to exchange the buffer. When the system was at equilibrium (buffer exchange complete) host protein contamination in the collected IgG (23.2 mg/mL) amounted to 153 ppm and aggregate content was 2.29%.

Example 16. The experimental steps of Example 15 were reproduced in a separate experiment with a different cell culture harvest, with other details as in Example 15 except as described below. Host protein content of the preparation going into the prototype apparatus was 196 ppm (versus the 490 ppm in Example 15). After buffer exchange to 50 mM Tris, pH 8.0, host contamination was reduced to 110 ppm. The adsorption subunit (QA monolith) was put in line, reducing host contamination to 40 ppm in 1 diavolume and to 26 ppm after 5 diavolumes.

Example 17. The experimental steps of Example 15 were reproduced in a separate experiment with a different cell culture harvest, with other details as in Example 15 except as described below. Host protein content of the preparation going into the prototype apparatus was 215 ppm (versus the 490 ppm in example 15). After buffer exchange to 50 mM Tris, pH 8.0, host contamination was reduced to 116 ppm. The adsorption subunit (QA monolith) was put in line, reducing host contamination to 28 ppm in 1 diavolume, and to 19 ppm after 5 diavolumes. The experiment was repeated with another aliquot of the same chromatin-deficient/HCX-processed antibody except substituting the QA monolith in the adsorbent subunit with a quaternary amine membrane adsorber (Sartobind Q, Sartorius). Host contamination was reduced to 28 ppm after 1 diavolume and to 17 ppm after 5 diavolumes. The experiment was repeated with another aliquot of the same chromatin-deficient/HCX-processed antibody except substituting the QA monolith in the adsorbent subunit with a column packed with a quaternary amine ion exchange chromatography particles (Nuvia Q, Bio-Rad Laboratories). Host contamination was reduced to 20 ppm after 1 diavolume and to 11 ppm after 5 diavolumes.

Example 18. Chromatin was extracted from an IgG-containing cell culture harvest with 260,183 ppm host protein contamination and 26.05% aggregates by treating it with 1% allantoin, 0.4% caprylic acid, pH 5.4; incubating it for 2 hours, removing the solids by microfiltration, then processing it by void exclusion anion exchange chromatography on a column of UNOsphere Q equilibrated to 50 mM Tris, pH 8.0. Host protein contamination was reduced to 228 ppm and aggregates to 2.47%. The preparation was applied to a column packed with electronegative multimodal particles (HCX, Merck-Millipore), the column washed, and the IgG was eluted by salt gradient, reducing host protein content to 38 ppm and aggregates to 1.93%. The preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). The adsorption subunit was in-line with the tangential flow filtration subunit as the buffer was exchanged from the original ˜0.7 M NaCl following HCX to 50 mM Tris, pH 8.0. Host protein contamination was reduced to 38 ppm and aggregates to 1.37%.

Example 19. In a parallel experiment, the steps of Example 18 were performed with a different cell culture harvest, initially containing 380,492 ppm host protein and 25.88% aggregates. Chromatin extraction reduced host protein levels to 1134 ppm and aggregates to 0.63%. HCX reduced host protein to 135 ppm and aggregates were at 0.82%. The preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). The adsorption subunit was in-line with the tangential flow filtration subunit as the buffer was exchanged from the original ˜0.7 M NaCl following HCX to 50 mM Tris, pH 8.0. Host protein contamination was reduced to 25 ppm and aggregates to 0.86%.

Example 20. Chromatin was extracted from an IgG-containing cell culture harvest with 260,184 ppm host protein contamination and 26.05% aggregates by treating it 1% allantoin, 0.4% caprylic acid, pH 5.4; incubating it for 2 hours, removing the solids by microfiltration, then passing it through a column packed with Bio-Works TREN (high) with a volume of 5% the volume of the original cell culture harvest in series with a depth filter (PB1, Sartorius), then processing it by void exclusion anion exchange chromatography on a column of UNOsphere Q equilibrated to 50 mM Tris, pH 8.0. Host protein contamination was reduced to 53 ppm and aggregates to 2.02%. The preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). Host protein contamination in the collected IgG amounted to 3 ppm and aggregate content was 1.76%. Application of the preparation to a column of Capto adhere (GE Healthcare) reduced host protein contamination below the level of detectability and aggregates to less than 0.1%.

Example 21. Chromatin was extracted from an IgG-containing cell culture harvest with 260,184 ppm host protein contamination and 26.05% aggregates by treating it 1% allantoin, 0.4% caprylic acid, pH 5.4; incubating it for 2 hours, removing the solids by microfiltration, then passing it through a column packed with Bio-Works TREN (high) with a volume of 5% the volume of the original cell culture harvest. Host protein was reduced to 4,122 ppm and aggregates to 3.32%. The preparation was processed by conventional cation exchange chromatography using Nuvia S (Bio-Rad Laboratories). Host protein was reduced to 34 ppm and aggregates to 2.69%. The preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). Host protein contamination was reduced to 6 ppm and aggregates to 1.75%. Application of the preparation to a column of Capto adhere (GE Healthcare) reduced host protein contamination below the level of detectability and aggregates to less than 0.1%.

Example 22. A pair of experiments was conducted in which an IgG-containing cell culture harvest was processed by protein A affinity chromatography to concentrate the antibody and reduce chromatin content. It contained 7,633 ppm host protein contamination. NaCl was added to a final concentration of 1 M. The preparation was divided into equal portion, and each was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong anion exchange monolith (CIM QA, BIA Separations). In the first experiment, the adsorption subunit was in-line with the tangential flow filtration subunit from the beginning. In the second experiment, the adsorption subunit was put in-line after the preparation had been cycled through the tangential flow filtration subunit for 5 diavolumes. Especially in the first example, the 1 M NaCl was understood to prevent contaminant binding to the adsorption unit and thereby promote alternative elimination of small contaminants through the pores of the membrane. After buffer exchange to 50 mM Tris pH 8.0, host protein was reduced to 399 ppm in the first experiment and 368 in the second.

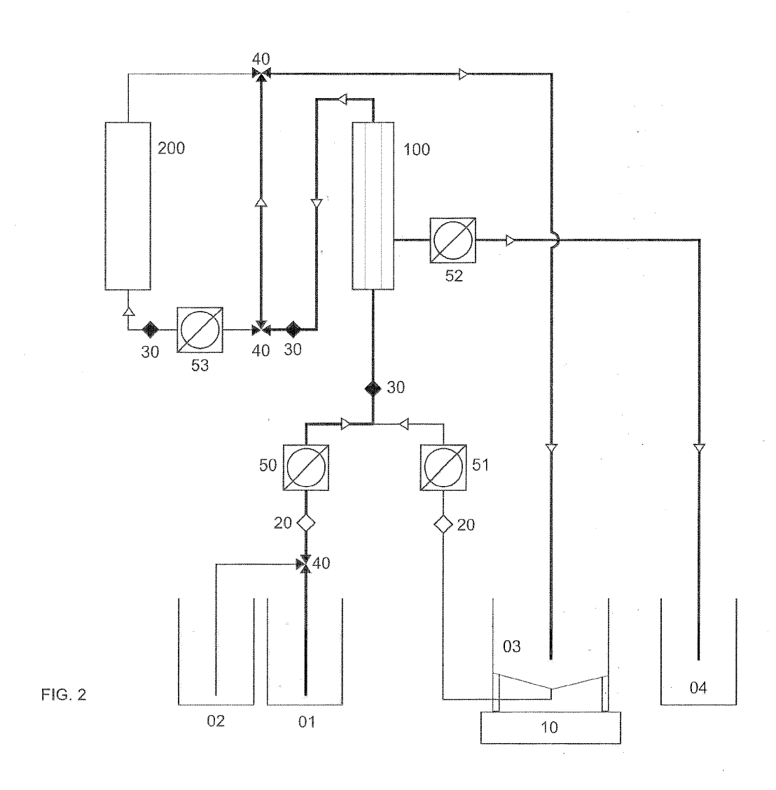

Example 23. In some experiments, chromatin deficient IgG-containing cell culture harvest was applied to a prototype apparatus where the adsorptive subunit was an electropositive membrane with porosity sufficient to retain IgG. Since such membranes are not available commercially, they were prepared according to the method of van Reis as described in World Patent WO20018792 A2, FIG. 2A. In brief, 30 kDa cellulose membranes (Sartorius 14459-76-D) were reacted with 2 M 3-bromopropyl trimethyl ammonium bromide at room temperature overnight. Distinct from van Reis, the membrane was then washed 1 M NaCl, then water, to eliminate residual reactants and reaction byproducts Immobilization of quaternary amino groups was confirmed by binding of the anionic dye Methyl Blue. Functionality of the electropositive membranes was demonstrated in brief as follows: Mammalian cell culture harvest containing an IgG monoclonal antibody at a concentration of about 1.5 g/L was clarified by adjustment to pH 5.4, addition of 1% allantoin, followed by 0.4% sodium caprylic acid, incubated stiffing for 2 hours. Solids were removed by microfiltration and the supernatant was flowed through a column containing agarose beads substituted with TREN (WorkBeads 40 TREN High, BioWorks, Uppsala), where the volume of the TREN column was 5% the volume of the original volume of cell culture harvest. This reduced host protein contaminants from the original 219,570 ppm to 4441 ppm. The sample was applied without equilibration to a 30 kDa quaternary amine substituted filter and diafiltered to 50 mM Tris, pH 8.0. Host protein contaminants were reduced to 679 ppm. NaCl was added to the processed IgG to a final concentration of 1 M, and applied to a column of Capto adhere (GE Healthcare) equilibrated to 50 mM Tris, 1 M NaCl, pH 8.0. The Capto adhere column was eluted with a step to 50 mM MES, 300 mM NaCl, pH 6.0. HCP content of the eluted IgG was less than 1 ppm, and contained less than 1 ppm DNA, and less than 0.1% aggregates.

Example 24. An IgM-containing mammalian cell culture harvest containing 644,254 ppm host protein contamination and 2.91% aggregate contamination was treated to remove chromatin by addition of caprylic acid to 0.4%, allantoin to 1%, then incubated for 2 hours at pH 5.4. TREN-bearing particles (TREN-high, Bio-Works) were added to a proportion of 5% and incubated for an additional 2 hours, following which solids were removed by centrifugation and the supernatant treated by void exclusion anion exchange chromatography on a column of UNOsphere Q equilibrated to 50 mM phosphate, 200 mM NaCl, pH 7.0. This removed more than 99% of the chromatin, as well as all the aggregates, and reduced host protein contamination to 13,881 ppm. The preparation was applied to a prototype apparatus configured with a single tangential flow filtration subunit equipped with a cellulose membrane with an average pore size corresponding to a hypothetical globular protein with a hydrodynamic diameter of 30 kDa (Millipore), and a single adsorption subunit with a strong cation exchange monolith (CIM SO3, BIA Separations). The preparation was buffer exchanged to 25 mM MES, 25 mM Hepes, pH 5.5 with the SO3 monolith in line, causing the IgM to bind to the monolith while the majority of small unadsorbed contaminants passed through the pores of the membrane. The monolith was then washed by buffer exchanging the preparation to 25 mM MES, 25 mM Hepes, pH 6.5; then the IgM was eluted by buffer exchanging the preparation to 25 mM MES, 25 mM Hepes, 125 mM NaCl, pH 6.5. The SO3 monolith was rinsed in-line with clean buffer then put off-line. The preparation was buffer exchanged to 50 mM Hepes, pH 7.0 then collected from the apparatus with 3,146 ppm host protein contamination and no aggregates. The preparation was applied to a QA monolith on a conventional chromatograph, washed and eluted, reducing host protein contamination to 29 ppm.

Example 25. An Escherichia coli cell culture harvest containing bacteriophage virus M13, 17,034 ng/mL of host protein contamination, and 0.24 ng/mL of host DNA was applied to a prototype apparatus configured with a single tangential flow filtration subunit equipped with a cellulose membrane with an average pore size corresponding to a hypothetical globular protein with a hydrodynamic diameter of 30 kDa (Millipore), and two adsorption subunits plumbed in parallel with respect to each other; plumbed in series with respect to the tangential flow filtration subunit, with valves to permit either or both of them to be put off line when desired. The adsorption subunits were equipped with a strong cation exchange monolith (CIM SO3) and a strong anion exchange monolith, (CIM QA). The preparation was buffer exchanged to 50 mM MES, pH 6.0 with the SO3 monolith in line. Under these conditions, the virus did not bind to the monolith but was retained by the membrane. Some contaminants were adsorbed to the SO3 monolith while small unadsorbed contaminants passed through the pores of the membrane. The preparation was buffer exchanged to rinse unadsorbed virus from the SO3 monolith. A sample collected at this point contained 243 ng/mL host protein contamination and 0.11 ng/mL DNA. Virus recovery at this point was 73%. The SO3 monolith was put off-line coincident with the QA monolith being put in-line. The buffer was exchanged to 50 mM Hepes, pH 7.0, during which the virus bound to the QA monolith, and during which small unadsorbed contaminants are understood to have passed through the pores of the membrane. The buffer was exchanged to 50 mM Hepes, 500 mM NaCl, pH 7.0 to elute the virus from the QA monolith, and the monolith was rinsed with clean buffer them put off line. The virus was collected from the system containing 90% ng/mL host protein and 0.04 ng/mL DNA. Virus recovery for this process segment was 86%, and virus recovery for the overall process was 63%.

The embodiments disclosed herein may be combined with other purification methods to achieve higher levels of purification. Some such embodiments may include performing the disclosed methods after some other fractionation method has been employed. Other such embodiments may include performing the disclosed methods before some other fractionation is to be employed. It is within the purview of a person of ordinary skill in the art to develop appropriate conditions for the various other methods and integrate them with the embodiments herein to achieve the necessary purification of a particular biological product.

It will be understood by persons of ordinary skill in the art that some benefits of the disclosed apparatus and methods can be obtained with variations of the disclosed apparatus and methods, including by uncoupling the tangential flow filtration subunit or subunits from the adsorbent chromatography subunit or subunits. Such uncoupled applications, such as any of the apparatus or process embodiments described herein where the preparation or other materials are conveyed between parts of the apparatus by means other than conduits including manual transfer by an operator from one portion to another portion of the apparatus are understood to be embodiments of the disclosed invention.

Persons of ordinary skill in the art will recognize that the above examples were designed to illustrate the effects of some of the most common variables and that many variations can be performed without departing from the essential elements of the disclosed apparatus and methods. Equally, it will be understood from that examples that different cell culture harvests contain different species at different relative preparations, which will impose substantial variability, and that further variability will be apparent among samples of a given preparation according to the method by which chromatin is extracted.

All references cited herein are incorporated by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

All numbers expressing quantities of ingredients, chromatography conditions, and so forth used in the specification and claims are to be understood as being modified in all instances by the term “about.” Accordingly, unless indicated to the contrary, the numerical parameters set forth in the specification and attached claims are approximations that may vary depending upon the desired performance sought to be obtained by the embodiments herein.

Many modifications and variations of the embodiments disclosed herein can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the embodiments being indicated by the following claims.

1. A process for purifying a biological product from a preparation comprising:
providing an apparatus comprising:
multiple purification subunits comprising a tangential flow filtration subunit equipped with at least one porous membrane having pores with a porosity sufficient to retain practically all of the biological product, and one or more adsorption subunits;
multiple conduits connecting the multiple purification units and associated pumps and valves, thereby allowing cycling of the biological product through the apparatus according to multiple alternate configurations including
(i) a first configuration for continuous flow such that the retentate line output of the tangential flow filtration subunit may be collected as recyclate and returned to the input of the tangential flow filtration unit, and
(ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and, optionally,
(iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit;
and conduits for supply of the preparation to the apparatus, preferably to the input of the tangential flow filtration unit;
performing Step A comprising operating the apparatus according to the first configuration such that the biological product may cycle through the tangential flow filtration subunit one or more times while increasing the concentration of the biological product and reducing the levels of contaminants associated with the biological product;
performing Step B comprising operating the apparatus according to the second configuration such that the biological product may cycle through the tangential flow filtration subunit and the adsorption subunit selected from the one or more adsorption subunits one or more times while reducing the levels of contaminants associated with the biological product;
and, optionally, performing Step C comprising operating the apparatus according to the third configuration such that the biological product may cycle through the tangential flow filtration subunit and the adsorption subunit different from the adsorption subunit selected in the second configuration one or more times while reducing the levels of contaminants associated with the biological product.

2. The process of claim 1 wherein (a) the apparatus provides for the first configuration, the second configuration and the third configuration and wherein each of Step A, Step B and Step C are performed; or (b) wherein the apparatus provides for the first configuration and the second configuration and further wherein Step A is performed followed by Step B and the purified biological product is obtained from the recyclate; or (c) the apparatus provides for the first configuration and the second configuration and further wherein Step A is performed followed by Step B, then Step A is performed again, and the purified biological product is obtained from the recyclate, and optionally in (c), the recyclate containing the purified biological product is flowed to an additional adsorption unit and the biological product exiting such additional adsorption unit is not returned to the tangential flow unit before exiting the apparatus.

3-6. (canceled)

7. The process of claim 1, wherein Step A is performed again between Step B and Step C, and the recyclate containing the purified biological product is flowed to an additional adsorption unit and the biological product exiting such additional adsorption unit is not returned to the tangential flow unit before exiting the apparatus.

8-9. (canceled)

10. The process of claim 1 wherein the provided apparatus comprises a second tangential flow filtration purification unit equipped with at least one porous membrane having pores with a porosity sufficient to retain practically all of the biological product, and a second adsorption unit, the multiple conduits additionally allow cycling of the biological product through the apparatus according to multiple alternate configurations including the first configuration, the second configuration and
(iv) a fourth configuration for continuous flow such that the retentate line output of the second tangential flow filtration subunit may be collected as recyclate and returned to the input of the second tangential flow filtration unit, and
(v) a fifth configuration for continuous flow such that the retentate line output of the second tangential flow filtration connects to the input for the second adsorption subunit such that the output of such adsorption unit may be collected as recyclate and returned to the input of the second tangential flow filtration unit;
and wherein the process further comprises
performing Step D comprising operating the apparatus according to the fourth configuration such that the biological product may cycle through the second tangential flow filtration subunit one or more times while increasing the concentration of the biological product and reducing the levels of contaminants associated with the biological product;
performing Step E comprising operating the apparatus according to the fifth configuration such that the biological product may cycle through the second tangential flow filtration subunit and the second adsorption subunit one or more times while reducing the levels of contaminants associated with the biological product.

11. The process of claim 10 wherein the apparatus provides for the first configuration, the second configuration, the fourth configuration and the fifth configuration and wherein each of Step A, Step B, Step D and Step E are performed.

12. The process of claim 1, wherein chemical conditions during portions of Step B, Step C or Step E comprise one or more of the following: (i) conditions preventing adsorption of the majority of the components of the preparation, (ii) conditions preventing or suspending adsorption of the biological product, and (iii) conditions permitting adsorption of the biological product.

13-22. (canceled)

23. The process of claim 10, wherein the first tangential flow subunit comprises a polyethersulfone membrane comprising 0.2 micron pores and the second tangential flow subunit comprises a cellulose membrane comprising pore sizes corresponding with a globular protein with a mass of 30 kDa.

24. The process of claims 10, wherein one or more membranes within one or more of the tangential flow filtration subunits has adsorptive surface characteristics such that it would be suitable for performing adsorption chromatography.

25. The process of claim 1, wherein a physical format of the membrane within the tangential flow filtration subunit is selected from the group consisting of a sheet, a wound (rolled) sheet, a hollow fiber, and combinations thereof; a physical format of the adsorptive subunit is selected from the group consisting of a column packed with adsorptive particles, a monolith, one or more adsorptive membranes, one or more sheets, one or more hollow fibers, and combinations thereof; and an adsorptive mechanism employed by the adsorptive subunit is independently selected from the group consisting of electrostatic interaction, hydrophobic interaction, pi-pi binding, hydrogen bonding, van der Waals interaction, metal affinity, biological affinity, and combinations thereof.

26-28. (canceled)

29. The process of claim 10, wherein the adsorption unit used in Step B, Step C or Step E is a monolith comprising an anion exchange adsorptive mechanism provided by quaternary amine moieties, a cation exchange adsorptive mechanism provided by SO3 moieties, or a hydrophobic interaction adsorptive mechanism provided by phenyl moieties.

30-39. (canceled)

40. The process of claim 10, wherein Step B is performed in a flow through mode and Step C or Step E is performed in a bind-elute mode.

41. (canceled)

42. The process of claim 1, wherein the biological products comprises a hydrodynamic diameter between 10 nm and 100 microns, or the biological product is selected from the group consisting of a DNA plasmid, a virus particle, a virus-like particle, a cellular organelle, a cell, an antibody, and a non-antibody protein.

43. (canceled)

44. The process of claim 42, wherein a source of the preparation comprises a cell culture harvest or a naturally occurring body fluid selected from the group consisting of serum, plasma, milk, and fluid from a tissue homogenate, and the preparation is provided in a form having less than 5% of the chromatin residing in a source sample from which the preparation was obtained.

45-47. (canceled)

48. An apparatus for purifying a biological product from a preparation comprising:
multiple purification subunits comprising a first tangential flow filtration subunit equipped with at least one porous membrane having pores with a porosity sufficient to retain practically all of the biological product and one or more adsorption subunits;
multiple conduits connecting the multiple purification units;
valves that direct flow through the multiple conduits and permit isolation of one or more of the multiple purification units from each other;
pumps configured to induce flow and control differential pressure within one or more portions of the apparatus;
and conduits for supply of the preparation to the apparatus, preferably to the input of the tangential flow filtration unit;
wherein the multiple conduits and associated pumps and valves allow cycling of the biological product through the apparatus according to multiple alternate configurations including
(i) a first configuration for continuous flow such that the retentate line output of the tangential flow filtration subunit may be collected as recyclate and returned to the input of the tangential flow filtration unit,
(ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and,
(iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit.

49. The apparatus of claim 48 further comprising a second tangential flow filtration purification unit equipped with at least one porous membrane having pores with a porosity sufficient to retain practically all of the biological product, and a second adsorption unit, wherein the multiple conduits additionally allow cycling of the biological product through the apparatus according to multiple alternate configurations including the first configuration, the second configuration, the third configuration and
(iv) a fourth configuration for continuous flow such that the retentate line output of the second tangential flow filtration subunit may be collected as recyclate and returned to the input of the second tangential flow filtration unit, and
(v) a fifth configuration for continuous flow such that the retentate line output of the second tangential flow filtration connects to the input for the second adsorption subunit such that the output of such adsorption unit may be collected as recyclate and returned to the input of the second tangential flow filtration unit.

50. An apparatus for purifying a biological product from a preparation comprising:
multiple purification subunits comprising a first tangential flow filtration subunit equipped with at least one porous membrane having an average pore size from about 2.5 nm to about 5000 nm, and one or more adsorption subunits;
multiple conduits connecting the multiple purification units;
valves that direct flow through the multiple conduits and permit isolation of one or more of the multiple purification units from each other;
pumps configured to induce flow and control differential pressure within one or more portions of the apparatus;
and conduits for supply of the preparation to the apparatus, preferably to the input of the tangential flow filtration unit;
wherein the multiple conduits and associated pumps and valves allow cycling of the biological product through the apparatus according to multiple alternate configurations including
(i) a first configuration for continuous flow such that the retentate line output of the tangential flow filtration subunit may be collected as recyclate and returned to the input of the tangential flow filtration unit,
(ii) a second configuration for continuous flow such that the retentate line output of the tangential flow filtration connects to the input for an adsorption subunit selected from the one or more adsorption subunits such that the output of such adsorption unit may be collected as recyclate and returned to the input of the tangential flow filtration unit; and,
(iii) a third configuration for continuous flow such that the retentate line output of the tangential flow filtration connects to the input for an adsorption subunit different from the adsorption subunit selected in the second configuration (and not through the adsorption subunit selected in the second configuration) such that the output of such adsorption unit may be collected as recyclate and returned to the input of the tangential flow filtration unit.

51. The apparatus of claim 50 further comprising a second tangential flow filtration purification unit equipped with at least one porous membrane having an average pore size from about 2.5 nm to about 5000 nm, and a second adsorption unit, wherein the multiple conduits additionally allow cycling of the biological product through the apparatus according to multiple alternate configurations including the first configuration, the second configuration, the third configuration and
(iv) a fourth configuration for continuous flow such that the retentate line output of the second tangential flow filtration subunit may be collected as recyclate and returned to the input of the second tangential flow filtration unit, and
(v) a fifth configuration for continuous flow such that the retentate line output of the second tangential flow filtration connects to the input for the second adsorption subunit such that the output of such adsorption unit may be collected as recyclate and returned to the input of the second tangential flow filtration unit.

52. The apparatus of claim 51, wherein the porous membranes of the tangential flow filtration subunits comprise an average pore size from about 5 nm to about 1000 nm; an average pore size selected to retain at least 99% of the biological product on the basis of its hydrodynamic radius; or an average pore size selected to be greater than the average hydrodynamic radius of the biological product.

53-59. (canceled)

60%. The apparatus of claim 51, wherein the membrane of the first tangential flow subunit comprises a polyethersulfone membrane comprising 0.2 micron pores and the membrane of the second tangential flow subunit comprises a cellulose membrane comprising pore sizes corresponding with a globular protein with a mass of 30 kDa.

61. The apparatus of claim 51, wherein one or more membranes within one or more of the tangential flow filtration subunits has adsorptive surface characteristics such that it would be suitable for performing adsorption chromatography.

62. The apparatus of claim 50, wherein a physical format of the membrane within the tangential flow filtration subunit is selected from the group consisting of a sheet, a wound (rolled) sheet, a hollow fiber, and combinations thereof; a physical format of the adsorptive subunits is selected from the group consisting of a column packed with adsorptive particles, a monolith, one or more adsorptive membranes, one or more sheets, one or more hollow fibers, and combinations thereof; and an adsorptive mechanism employed by the adsorptive subunits is independently selected from the group consisting of electrostatic interaction, hydrophobic interaction, pi-pi binding, hydrogen bonding, van der Waals interaction, metal affinity, biological affinity, and combinations thereof.

63-64. (canceled)

65. The apparatus of claim 50, wherein the adsorption unit used in the second or third configuration is a monolith having an anion exchange adsorptive mechanism provided by quaternary amine moieties; or a monolith having a cation exchange adsorptive mechanism provided by SO3 moieties.

66-70. (canceled)

71. The apparatus of claim 51, wherein the adsorption unit used in the fifth configuration is a monolith having an anion exchange adsorptive mechanism provided by quaternary amine moieties; or a monolith having a hydrophobic interaction adsorptive mechanism provided by phenyl moieties.

72-76. (canceled)

77. The apparatus of claim 50, further comprising one or more processors configured by computer readable instructions to control the pumps and valves to pass the preparation through a fluid path in accordance with path instructions, wherein the path instructions define a sequence of purification units.

78. The apparatus of claim 77, further comprising an interface configured to receive entry or selection by a user of path instructions.

79-82. (canceled)
